(19) United States
(12) Patent Application Publication
(10) Pub. No.: US 2008/0275112 A1
(43) Pub. Date: Nov. 6, 2008

(54) Invention Concerning Aminoadamantane Compounds

(76) Inventors: Peter R. Schreiner, Wettenberg (DE); Lukas Wanka, Wollstadt (DE)

Correspondence Address: CLARK & BRODY, 1090 VERMONT AVENUE, NW, SUITE 250, WASHINGTON, DC, 20005, US

(21) Appl. No.: 11/658,172

(22) PCT Filed: Jul. 22, 2005
(86) PCT No.: PCT/DE2005/001304

§ 371 Date: Feb. 29, 2008

(30) Foreign Application Priority Data
Jul. 23, 2004 (DE) 10 2004 035 978.4
Jul. 8, 2005 (DE) 10 2005 032 380.4

(51) Int. Cl. A61K 31/27; C07C 233/08; C07C 211/38; C07C 23/38; A61K 31/16; A61K 31/025; A61P 25/00; A61P 33/00; A61K 31/215; A61K 31/196; A61K 31/13; C07C 61/135; C07C 271/24; C07C 229/50

(52) U.S. Cl. 514/480; 564/217; 564/459; 570/187; 562/499; 560/115; 560/117; 514/629; 514/662; 514/755; 514/563; 514/561; 514/511

(57) ABSTRACT

The present invention describes 1-aminoadamantane derivatives and 3-aminoadamantane-1-carboxylic derivatives in which the 5- or 7-position of the basic adamantane structure can be optionally substituted, methods for the production of the compounds based on the present invention, and methods for the coupling of the monomeric 3-aminoadamantane-1-carboxylic derivatives thus obtained to oligomers. The compounds based on the present invention are suitable for the utilisation as antiviral active ingredients, artificial ion channels, as well as for the therapy, diagnostics and prophylaxis of diseases in which a dysfunction of the GABA system occurs.

The present invention concerns novel aminoadamantane derivatives as well as methods of synthesis for the production of aminoadamantane derivatives. The novel methods of synthesis, based on the present invention, allow, for the first time, the production of aminoadamantane derivatives in which a tertiary H atom of the adamantane skeleton has been substituted by an amino group and at least one, or a maximum of all three of the other tertiary H atoms have been substituted by a number of functional groups. Of particular preference, at this juncture, are the production of 3-aminoadamantane-1-carboxylic acid derivatives in which position 5 or 7 of the adamantane skeleton has been optionally substituted as well as the coupling of the monomeric amino acid derivatives, obtained in this manner, to oligomers.

The present invention concerns the fields of chemistry, biochemistry, biology, and pharmacology.

In the current technical state of the art, several methods are known for the production of adamantane derivatives. The bromination of adamantane at the bridgehead is described, for instance, in: P. R. Schreiner, O. Lauenstein, I. V. Kolomytsin, S. Nadi, A. A. Fokin: Selective C—H-Activation of Aliphatic Hydrocarbons under Phase-Transfer-Conditions, Angew. Chem. Int. Ed. 1998, 37, 1895-1897. Methods for the iodination of the adamantane's bridgehead C atom are described in: P. R. Schreiner, O. Lauenstein, E. D. Butova A. A. Fokin: The First Efficient Iodination of Unactivated Aliphatic Hydrocarbons, Angew. Chem. Int. Ed. 1999, 38, 2786-2788 and in DE 198 44 865 C1 . Methods for the halogenation of one or several tertiary C atoms of adamantane under phase transfer conditions are described in: A. A. Fokin, O. Lauenstein, P. A. Gunchenko, P. R. Schreiner: Halogenation of Cubane under Phase-Transfer Conditions: Single and Double C—H-Bond Substitution with Conservation of the Cage Structure, J. Am. Chem. Soc. 2001, 123, 1842-1847, as well as in: P. R. Schreiner, O. Lauenstein, E. D. Butova, P. A. Gunchenko, I. V. Kolomitsin, A. Wittkopp, G. Feder, A. A. Fokin: Chem. Eur. J. 2001, 7, 4996-5003. Radicalic substitutions to adamantane are described in: A. A. Fokin, P. R. Schreiner: Selective Alkane Transformations via Radicals and Radical Cations: Insights into the Activation Step from Experiment and Theory, Chem. Rev. 2002, 102, 1551-1593. The aforementioned methods for the halogenation of adamantane do not result in 3-aminoadamantane-1-carboxylic acid derivatives. A modified Ritter reaction for the synthesis of amides from aryl halides or alkyl halides is described in: G. A. Olah, B. G. B. Gupta, S. C. Narang: Synthetic Methods and Reactions; 66: Nitrosonium Ion Induced Preparation of Amides from Alkyl (Arylalkyl) Halides with Nitriles, a Mild and Selective Ritter-Type Reaction, Synthesis 1979, 274-276. Methods for the synthesis of 3-aminoadamantane-1-carboxylic acids are described in: F. N. Stepanov, Y. T. Srebrodolskii: Zhurnal Organicheskoii Khimii 1966, 2(9), 1612-1615; F. N. Stepanov, Y. I. Srebrodolskii: Khimicheskoe Mashinostroenie i Tekhnologiya 1966, 2, 6-10, and S. S. Novikov, A. P. Khardin, L. N. Butenko, I. A. Kulev, I. A. Novakov, S. S. Radchenko, S. S. Burdenko: Zhurnal Organicheskoii Khimii 1980, 16 (7), 1433-1435.

Adamantane derivatives are utilised, for instance, in pharmacology and in phytosanitary measures. The suitability of some adamantane derivatives as endothelin, neurokinin, or angiotensin antagonists or as antiviral agents is known. The antiviral properties of 3-aminoadamantane-1-carboxylic acid regarding its effectiveness against influenza have been described in: Neth. Appl (1966), 8 pp. CODEN: NAXXAN NL 6600715 19660721 CAN 66:2279 AN: 1967:2279. DE 696 26 650 T2 describes the utilisation of an aminoadamantane compound for the production of a drug against agranulomatosis, in which at least one of the four tertiary C atoms of adamantane carries an amino group and the other three tertiary C atoms have optionally been substituted by amino, alkyl, or aryl groups. DE 691 30 408 T2 describes peptide derivatives as antagonists of the endothelin receptor in which the C-terminal amino acid carries a 1-alkyl adamantyl group in α-position. DE 690 02 950 T2 describes peptides with a non-cleavable transition state insert corresponding to the 10,11 position of a rennin substrate (angiotensinogen) in which the insert can feature a 1- or 2-adamantyl group as well as a secondary amino group in a geminal position. In DE 44 06 884 A1 and DE 44 06 885 A1, amino acid derivatives for the therapy and prophylaxis of Neurokinin-mediated diseases are described in which a natural or non-natural amino acid is bound C-terminally to the amino group of a 3-amino-4-dihydro-1H-quinoline derivative and bound N-terminally to a ω-adamantyl-1-yl-alkane carboxylic acid via an amide bond. In addition, amino acid derivatives for the therapy and prophylaxis of Neurokinin-mediated diseases are described in DE 195 41 283 A1, in which the N-terminal amino acid can be connected to a 1-carboxyl-3-acetamido-adamantyl group; furthermore, the adamantyl group may be optionally OH substituted in the 5- or 7-position. DE 196 26 311 A1 describes 3-amino-2-hydroxy benzoic acid derivatives (for pest control in plants and technical material), of which the amino group is connected to a further carboxy group and the carboxy group of which is connected to a further amino group, resulting, in each case, in a carboxylic acid amide; the further amino group can be a 1-carboxy-3-amino-5,7-substituted adamantane derivative.

DE 2 318 461 describes 1N-alkyl substituted 3,5-dialkyl-1-aminoadamantane derivatives and their production. According to DE 2 318 461, the effect of 1-aminoadamantane on the human and animal central nervous system and the utilisation of this substance in the treatment of Morbus Parkinson is known. These 1N-alkyl substituted 3,5-dialkyl-1aminoadamantane derivatives are suited for the treatment of parkinsonism and other hyperkineses such as head tremor, thalamic syndrome, and spastic states; additionally, for the activation of akinetic states of cerebral organic origin. They influence the spiroperidol catalepsy and the antagonism against reserpine sedation.

The effect of aminoadamantane derivatives against protozoans of the genus Plasmodium is described, for example, in U.S. Pat. No. 6,737,438 B2, in U.S. Pat. No. 6,825,230 B2, in WO 2003/076425 A1, in EP 0 370 320 B1 and in U.S. Pat. No. 6,486,199 B1. Plasmodia represent the causative organisms of malaria. A person skilled in the art knows of four species of plasmodia causing malaria in humans: Plasmodium falciparum, Plasmodium ovale, Plasmodium vivax, and Plasmodium malariae. Further plasmodia species known to the skilled person are, for example, Plasmodium yoeli nigeriensis, Plasmodium vinckei petteri, Plasmodium berghei yoelii, Plasmodium gallinaceum, Plasmodium gallinaceum II and Plasmodium relictum. The antiprotozoic effect of aminoadamantane derivatives against some plasmodia not causing human malaria is furthermore described in the patent applications cited above.

To date, the current technical state of the art only knows methods for the synthesis of adamantane derivatives, the reaction conditions of which restrict the range of introducible functional groups considerably. Only few 3-, 3,5- and 3,5,7-substituted aminoadamantane derivatives are known. Particularly from the group of pharmacologically relevant 3-aminoadamantane-1-carboxylic acids, only a small number of theoretically possible acids have been producible in reality. Presently, oligopeptides of these latter compounds are completely unknown. Thus, dibromoadamantane, for instance, can be produced in yields of 95 to 98% via the reaction of adamantane with bromine and iron filings.

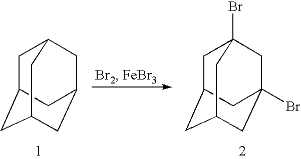

This process is known to the person skilled in the art and can be consulted, for example, in: T M Gorrie, P von Ragué Schleyer: Preparation of 1,3-dibromoadamantane. In: Organic Preparations and Procedures International 1971, 3(3), 159-162, as well as: I. R. Likhotvorik, N. L. Dovgan, G. I. Danilenko, Zhurnal Organicheskoi Khimii 1977, 13, 897-897, and: N. L. Dovgan, I. R. Likhotvorik, Selective Dibromination of Adamantane. Vestn. Kiev. Politekhn. In.-ta. Khim. Mashinostr. i. Tekhnol. (1979), (16), 20-22. The manufacturing of mixed dihalides of adamantane via this process, however, is not possible.

Alternatively, adamantane derivatives can also be halogenated one or more times via phase transfer catalysis, hereinafter referred to as PTC.

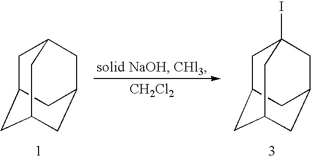

The production of 1-iodo-adamantane via PTC is known to the person skilled in the art and has been described, for example, in: P. R. Schreiner, O. Lauenstein, E. D. Butova, A. A. Fokin: The first efficient iodination of unactivated aliphatic hydrocarbons, Angew. Chem. Int. Ed. 1999, 38, 2786-2788. The production takes place by dissolving adamantane in methylene chloride and allowing this solution to react with iodoform and solid NaOH.

It is known to the person skilled in the art that this PTC iodination also works regioselectively in adamantane derivatives, for instance, in ethers, non-enolisable ketones, phenyl and phenoxy derivatives of the adamantane. This has been described in: P. R. Schreiner, O. Lauenstein, E. D. Butova, P. A. Gunchenko, I. V. Kolomitsin, A. Wittkopp, G. Feder, A. A. Fokin: Selective radical reactions in multiphase systems: phase-transfer halogenations of alkanes, Chem. Eur. J. 2001, 7, 4996-5003.

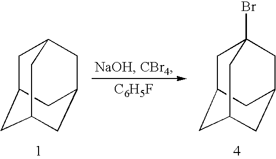

The production of 1-bromo-adamantane via PTC is known to the person skilled in the art and has been described, for example, in: P. R. Schreiner, O. Lauenstein, E. D. Butova, P. A. Gunchenko, I. V. Kolomitsin, A. Wittkopp, G. Feder, A. A. Fokin: Selective radical reactions in multiphase systems: phase-transfer halogenations of alkanes, Chem. Eur. J. 2001, 7, 4996-5003. Thereby, adamantane is dissolved in fluorobenzene and reacts with aqueous NaOH with a concentration of 10-50%, tetrabromomethane, and catalytic quantities of tetra-(n-butyl)-ammoniumbromide. It is known to the person skilled in the art that this PTC bromination—in the same way as PTC iodination—also works regioselectively in adamantane derivatives, for instance, in ethers, non-enolisable ketones, phenyl and phenoxy derivatives of the adamantane.

The manufacturing of 1,3-dihalosubstituted adamantane derivatives by a two-fold reaction with a halogen source in a biphase system under exposure of a phase transfer catalyst is known to the person skilled in the art and has been described in: P. R. Schreiner, O. Lauenstein, I. V. Kolomytsin, S. Nadi, A. A. Fokin: Selective C-H-Activation of Aliphatic Hydrocarbons under Phase-Transfer-Conditions, Angew. Chem. Int. Ed. 1998, 37, 1895-1897; P. R. Schreiner, O. Lauenstein, E. D. Butova A. A. Fokin: The First Efficient Iodination of Unactivated Aliphatic Hydrocarbons, Angew. Chem. Int. Ed. 1999, 38, 2786-2788; DE 198 44 865 C1; A. A. Fokin, O. Lauenstein, P. A. Gunchenko, P. R. Schreiner: Halogenation of Cubane under Phase-Transfer Conditions: Single and Double C—H-Bond Substitution with Conservation of the Cage Structure, J. Am. Chem. Soc. 2001, 123, 1842-1847, P. R. Schreiner, O. Lauenstein, E. D. Butova, P. A. Gunchenko, I. V. Kolomitsin, A. Wittkopp, G. Feder, A. A. Fokin: Chem. Eur. J. 2001, 7, 4996-5003 as well as in: A. A. Fokin, P. R. Schreiner: Selective Alkane Transformations via Radicals and Radical Cations: Insights into the Activation Step from Experiment and Theory, Chem. Rev. 2002, 102, 1551-1593.

It is known to the person skilled in the art that the order of reactivity of the halogens allows for the production of mixed dihalides in the order I>Br>Cl>F.

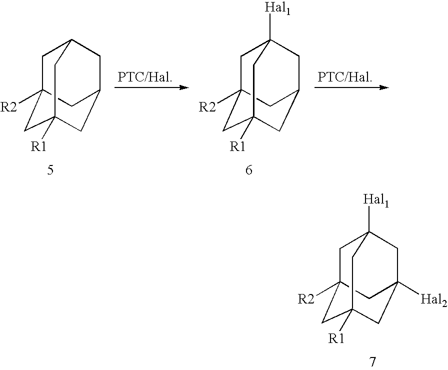

The conversion of halogenoadamantanes to alkyl adamantanes is made possible by the reaction of halogenoadamantane derivatives with Grignard reagents and/or lithium organyles, as described in: K. Takeuchi, T. Okazaki, T. Kitagawa, T. Ushino, K. Ueda, T. Endo, R. Notario: Influence of Alkyl Substitution on the Gas-Phase Stability of 1-Adamantyl Cation and on the Solvent Effects in the Solvolysis of 1-Bromoadamantane. J. Org. Chem. 2001, 66(6), 2034-2043 and G. Molle, J. E. Dubois, P. Bauer, Can. J. Chem. 1987, 65, 2428.

Suitable methods for the reaction of lithium organyles with alkyl halides are known to the person skilled in the art and can be consulted, for example, in: F. R. Hartley, S. Patai, eds., “The Chemistry of the Metal-Carbon-Bond”, Wiley: N.Y., 1982, 1985, 1986, Vol. 3: Carbon-Carbon Bond Formation using Organometallic Compounds.

The introduction of an amino group is made possible, for instance, by a reaction combined with the heating of halogenoadamantane derivatives with acetamide, yielding the corresponding acetamidoadamantane compound and its basic hydrolysis in polyethylenglycole solution containing KOH, as described in: J. G. Henkel, J. T. Hane, Structure-Anti-Parkinson Activity Relationships in the Aminoadamantanes. Influence of Bridgehead Substitution, J. Med. Chem. 1982, 25, 51-56. An alternative method can be found in: A. Jirgensons, V. Kauss, I. Kalvinish, M. R. Gold, Synthesis, 2000, 12, 1709-1712.

Via the reaction of the halogenoadamantanes into Grignard compounds, as described in:. G. Molle, P. Bauer, J. E. Dubois, Formation of Cage-Structure Organomagnesium Compounds. Influence of the Degree of Adsorption of the Transient Species at the Metal Surface, J. Org. Chem. 1982, 47, 41204128, and subsequent synthesis steps, a variety of other functionalisations are possible. Among others, the following can be introduced:
a.) —COOH via a reaction with dry CO₂;
b.) —CO—R7 via a reaction with carboxylic esters R7—COOR8;
c.) —CH(OH)—R9 via a reaction with aldehydes R9—CHO;
d.) —CR10R11OH via a reaction with ketones R10—CO—R11;
e.) —CH₂OH via a reaction with formic acid H₂C═O; in this context, R7—R11 denounces alkyl-, cycloalkyl-, aryl-, alkenyl- and alkinyl groups as they are listed in the chapter “Aim of the invention” for R4.
   All of these procedures are known to the person skilled in the art and can be consulted in textbooks such as: H. Beyer, Lehrbuch der Organischen Chemie, 23rd edition, Stuttgart, Leipzig: Hirzel 1998.
   The reaction of iodoadamantanes with alkali sulphite enables the approach to the corresponding sulphonic acids; their reaction with phosphorus(V)chloride and subsequent treatment with ammonia or amines provides an approach to sulfonamides. These reactions are known to the person skilled in the art and can be consulted, for example, in: H. Beyer, Lehrbuch der Organischen Chemie, 23rd edition, Stuttgart, Leipzig: Hirzel 1998.

Reactions of halogenoadamantanes to corresponding hydroxyadamantanes have been described in: F. N. Stepanov, Y. I. Srebrodol'skii, Zhurnal Organicheskoi Khimii 1966, 2(9), 1633-1634. Via esterification according to standard procedures as can be consulted, for instance, in: H. G. O. Becker, Organikum: organisch-chemisches Grundpraktikum, 20^{th} edition, Heidelberg, Leipzig: Barth 1996, the introduction of carboxylic acid residues, particularly fatty acid residues, is possible.

According to Sasaki et al. (cf. T. Sasaki, A. Nakanishi, M. Ohno, J. Org. Chem. 1981, 46 (26) 5445-5447; T. Sasaki, A. Usuki, M. Ohno, J. Org. Chem. 1980, 45 (18) 3559-3564 and T. Sasaki, A. Usuki, M. Ohno, Tetrahedron Lett. 1978, 49, 4925-4928), a variety of further substituents is introducible and may have to be further modified by catalytic hydrogenation. Some of these substituents are listed here as examples, but not exhaustively:

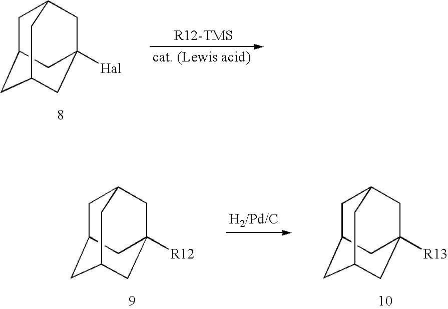

Here, the halogenoadamantane is treated with allyltrimethylsilane or its heteroanalogues in the presence of Lewis acids such as TiCl₄ or AlCl₃ and the resulting product may be subjected to heterogeneous catalysed hydrogenation if necessary. Thus, the following are examples of possible reactions but not exhaustive; TMS represents “trimethylsilyl”.

| R12-TMS | R12 | R13 |
|---|---|---|
| 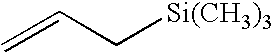 | 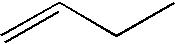 | 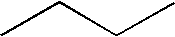 |
| 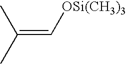 | 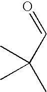 | 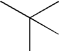 |
| 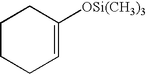 | 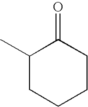 | 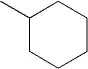 |
| 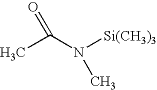 | 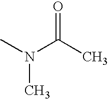 | — |
| 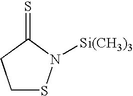 | 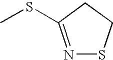 | — |
| 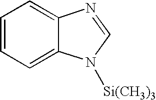 | 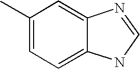 | — |
| 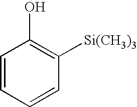 | 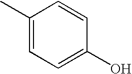 | — |
| 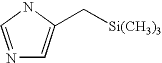 | 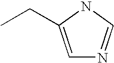 | — |
| 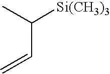 | 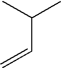 | 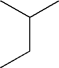 |
| 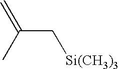 | 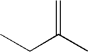 | 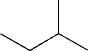 |
| 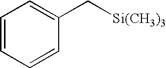 | 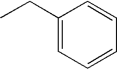 | — |
| 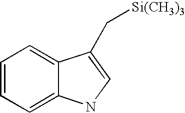 | 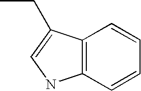 | — |
| 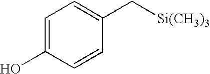 | 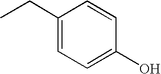 | — |

R12, R13 can be, as listed in the table above, e.g. prop-2-enyl, 2-methyl-prop-1-enyl, cyclohex-1-enyl, N-acetyl-N-methylamino, (2-isothiazolidin-3-thionyl)methyl (or respectively after the reaction, 3-(4,5-dihydro-isothiazolyl)sulfanyl-, 3-(1H-benzoimidazolyl)methyl (or respectively after the reaction, 5-(1H-benzoimidazolyl)methyl), 2-hydroxyphenyl (or respectively after the reaction, 4-hydroxyphenyl), 5-(1H-imidazolyl)methyl, 3-but-1-enyl, 3-(2-methyl)prop-1-enyl, benzyl, 3-(1H-indolyl)methyl, 4-hydroxyphenylmethyl; R13 may be, among others, propyl, 1,1-dimethylethyl (tert.-butyl), cyclohexyl, 2-butyl or 2-methylpropyl.
It is known to the person skilled in the art, e.g. from the publications mentioned above, which combinations of Lewis acids, reaction conditions and reaction time must be used to produce the compounds 6 and 7 listed here. It is also known to the person skilled in the art that he/she can use further trimethylsilyl compounds to introduce aliphatic, aromatic, and araliphatic residues, in which these residues are linear, cyclic, and/or branched, and in which up to five carbon atoms are substituted by hetero atoms selected from one of the following: sulphur, nitrogen, or phosphorus.
Via the reaction of the halogenoadamantanes with saline cyanides, one can also arrive at adamantyl nitrites. This reaction is made possible, for instance, according to: J. Applequist, P. Rivers, D. E. Applequist, Theoretical and Experimental Studies of Optically Active Bridgehead-Substituted Adamantanes and Related Compounds, J. Am. Chem. Soc. 1969, 91, 5705-5711, S. Kim, H. J. Song, Tin-Free Radical Cyanation of Alkyl Iodides and Alkyl Phenyl Tellurides, Synleft, 2002, 12, 2110-2112 or G. S. Lee, J. N. Bashara, G. Sabih, A. Oganesyan, G. Godjoian, H. M. Duong, E. R. Martinez, C. G. Gutierrez, Photochemical Preparation of 1,3,5,7-Tetracyanoadamantane and Its Conversion to 1,3,5,7-Tetrakis(aminomethyl)adamantane, Org. Left. 2004, 6(11), 1705-1707. Via the following reactions known to the person skilled in the art, it is therefore possible, among other things, to introduce
a.) carbamoyl substitution,
b.) carboxy groups and, in further reactions, esters thereof,
c.) aminomethyl groups,
d.) aldehyde groups,
e.) keto groups,
f.) ester groups, or
g.) further amide groups.
   Procedures for these reactions can be found, for instance, in: H. G. O. Becker, Organikum: organisch-chemisches Grundpraktikum, 20th edition, Heidelberg, Leipzig: Barth 1996; references to the original publication can be found, for instance, in: R. C. Larock, Comprehensive Organic Reactions, Weinheim: V C H 1989.
   DE 23 18 461 describes a procedure for the production of 3,5-dialkyl substituted 1-aminoadamantanes in which 3,5-dialkyl substituted 1-halogenoadamantane compounds react with urea or an N,N′-dialkyl urea derivative at temperatures of 120° C. to 260° C. and subsequently, by cleavage of the crude amide thus obtained with concentrated mineral acids, the corresponding amine is released. The 3,5-dialkyl-1-halogenoadamantanes to be employed as educts are preferentially the chlorinated or brominated derivatives.

The disadvantage of this process is that halogenated adamantane compounds have to be utilised and numerous reaction steps must be carried out. Due to the necessity of having halogenoadamantanes and urea derivatives react with each other at high temperatures, this procedure is also very energy-consuming.

Due to the reaction conditions connected to them, the production methods of the current technical state of the art limit the attainable degree of substitution of the adamantane and/or the selection of the introducible functional groups. The present invention, in contrast, provides methods for the production of adamantane derivatives which allow for a higher degree of substitution as well as a greater selection of introducible functional groups. In this way, for the first time, the production of peptides based on 5,7-substituted monomeric and oligomeric 3-aminoadamantane-1-carboxylic acids is also possible.

Cyclic peptides play a central role in the construction of artificial ion channels. Artificial ion channels are known to the person skilled in the art and have been described, for instance, in: N. Voyer, M. Robitaille: “A novel functional artificial ion channel”, J. Am. Chem. Soc. 1995, 117, 6599-6600 and V. Sidorov, F. W. Kotch, J. L. Kuebler, Y. F. Lam, J. T. Davis: “Chloride transport across lipid bilayers and transmembrane potential induction by an oligophenoxyacetamide”, J. Am. Chem. Soc. 2003, 125, 2840-2841. To be able to form a channel structure, these artificial ion channels must have certain key characteristics: They must span the lipid layer of the cell membrane and be amphiphilic, i.e. have both polar and hydrophobic sections. This amphiphilicity directs the polar “head groups” towards the outer aqueous environment of the membrane while the hydrophobic region anchors in the cell membrane. Many of the hitherto known artificial ion channels consist of comparatively simple and repetitive molecule units. The decisive factor for the suitability as an artificial ion channel is the pore diameter of these molecules because it influences the ion selectivity of the channel and allows the passage of ions, e.g. potassium, sodium, calcium, or chloride ions, by size exclusion. In the ideal case, such an ion channel favours a particular ion, in which the ion's rate of active transport through the channel should be in the range of 10⁴ up to 10⁸ ions per second.

Currently, two approaches are being pursued in the production of artificial ion channels: In the first one, helical molecules are utilised, in which the ion channel forms either within a helix or between two interlinked helices. This concept is referred to as a protein-based or helical (ion) channel. Alternatively, there are compounds whose selectivity for particular alkali ions is known. These compounds are utilised as selective filters and combined with membrane-spanning molecules, in which the latter direct the ions towards and away from the selective filters. This concept is referred to as an ionophore-based ion channel. Both concepts are known to the person skilled in the art and can be consulted, for instance, in: P. J. Cragg, “Artificial transmembrane channels for sodium and potassium”, Science Progress 2002, 85, 219-241.

Artificial ion channels are also described in: T. D. Clark, L. K. Buehler, M. R. Ghadiri, J. Am. Chem. Soc. 1998, 120, 651-656, M. R. Ghadiri, J. R. Granja, R. A. Milligan, D. E. McRee, N. Khazanovich, Nature 1994, 372, 709-709, T. D. Clark, M. R. Ghadiri, J. Am. Chem. Soc. 1995, 117, 12364-12365, and K. Motesharei, M. R. Ghadiri, J. Am. Chem. Soc. 1997, 119, 11306-11312.
They are of interest, for instance, in the therapy of cystic fibrosis and mucoviscidosis because patients suffering from one of these hereditary diseases do not have functioning chloride channels and artificial, chloride-transporting ion channels could replace the missing native channels.
To date, the technical state of the art does not know any ion channels containing 5,7-substituted 3-aminoadamantane-1-carboxylic acid derivatives.

Peptidic catalysts and their utilisation for pharmaceutical purposes are known to the person skilled in the art and have been described, for instance, in: M. M. Vasbinder, E. R. Jarvo, S. J. Miller, Angew. Chem. Int. Ed. 2001, 40, 2824-2827 und F. Formaggio, A. Barazza, A. Bertocco, C. Toniolo, Q. B. Broxterman, B. Kaptein, E. Brasola, P. Pengo, L. Pasquato, P. Scrimin, J. Org. Chem. 2004, 69, 3849-3856. Currently, however, mainly such peptidic catalysts are known which are essentially based on chain-like, oligomeric α-amino acids in the D or L configuration. Peptides consisting of L-amino acids, however, are easily cleaved by proteases, while chain-like D-amino acids are potentially allergenic. Presently, no peptidic catalysts which contain amino acids with a cage structure are known.

The simplest γ-amino acid, γ-amino butyric acid (GABA), is the most important inhibiting neurotransmitter in the central nervous system. The GABA-mediated neuronal transmission is ended within a few milliseconds by the uptake of the neurotransmitter via specific, highly affine GABA transporters. It is known to the person skilled in the art that currently, four GABA-selective transporters are known in mammals which have a uniform structure and form a sub-family of the electrogenic Na⁺/Cl⁻ neurotransmitters. GABA transporters can be found in all parts of the brain, i.e. for example, in the hippocampus, the hypothalamus, the medulla, and the prefrontal cortex, furthermore in the spinal cord and most inhibitory synapses of the nervous system. This is known to the person skilled in the art and can be consulted, for example, in: L. A. Borden, K. E. Smith, P. R. Hartig, T. A. Branchek, R. A. Weinshank, Molecular heterogeneity of the gamma-aminobutyric acid (GABA) transport system. J. Biol. Chem. 1992, 267, 21098-21104. Disorders of the GABA metabolism lead to various diseases, among them, for example, Morbus Parkinson, Chorea Huntington, Morbus Alzheimer, autism, Tourette syndrome, hypertension, sleep disorders, ADHD, psychoses, panic and anxiety disorders, posttraumatic stress syndrome, bipolar affective disorders such as manic depressive disorders, schizophrenia. In disturbances of the GABA-mediated neuronal transmission, a delivery of GABA or similar γ-amino acids is therefore desirable. In comparison to GABA itself and analogues corresponding to the technical state of the art, the 3-aminoadamantane-1-carboxylic acids based on the present invention are considerably more lipophilic, due to the cycloaliphatic adamantane core, which facilitates the overcoming of the blood-brain barrier and fixates in regard to the three-dimensional orientation of the functional groups, particularly the carboxy-resp. amino function.

The aim of the present invention is to provide compounds containing at least one 1-aminoadamantane derivative, wherein this 1-aminoadamantane derivative contains a functional group different from hydrogen in the 3- and/or 5- and/or 7-position of the adamantane skeleton. This aim is achieved, according to the present invention, by the characteristics of claim 1 and subordinate claims 2 to 9.

One further aim of the invention is to provide methods for the production of these substances and for the oligomerisation of the monomeric 5,7-substituted 3-aminoadamantane-1-carboxylic acids obtainable in this way, wherein the oligomers are linear or cyclic. This aim is achieved, according to the present invention, by claims 10 to 15.

The present invention overcomes the disadvantages of the current technical state of the art by providing a number of 3, 3,5- and 3,5,7-substituted aminoadamantane compounds. In addition, the present invention provides processes for
producing 3- and 3,5-substituted aminoadamantane compounds based on the present invention by direct acetamidation and
producing 5,7-substituted 3-aminoadamantane-1-carboxylic acids based on the present invention and to combine them to form oligomers.
   The direct production of carboxylic acid amides, based on the present invention, avoids the prior halogenation (preferentially bromination) of the R14, R15-substituted adamantane and thereby saves one synthesis step. The chemicals needed for this reaction (HNO₃, H₂SO₄, HCl, oleum, nitrile) are inexpensive and available in large quantities. The method based on the present invention completely saves completely the costs for the disposal of halogen-containing (preferentially bromine-containing) waste.

The monomeric and oligomeric 5,7-substituted 3-aminoadamantane-1-carboxylic acid derivatives based on the present invention are suited as antiviral agents, GABA analogues, persistent oligopeptides, creators of artificial ion channels, and modules for peptidic catalysts.

The compounds based on the present invention which are substituted in the 3- and/or 5- and/or 7-position of the 1-aminoadamantane skeleton have the general structural formula

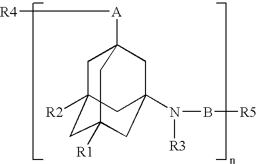

wherein R1 and R2 represent, independently from each other:

### H, F, Cl, Br, I;

L=-alkyl, -alkenyl, -alkinyl, -cycloalkyl, -cycloalkenyl, -heterocycloalkyl, -heterocycloalkenyl, -aryl, -heteroaryl, -alkylaryl, -alkylheteroaryl, -alkylcycloalkyl, -alkylheterocycloalkyl, -alkenylcycloalkyl, -alkenylheterocycloalkyl,
wherein -alkyl represents a group containing 1 up to 10 carbon atoms, preferentially methyl, ethyl, propyl, isopropyl, 1-butyl, 2-butyl, (2-methyl)propyl, tert.-butyl and
-alkenyl and -alkinyl represent a monounsaturated or polyunsaturated group with 2 up to 12 carbon atoms which, in the case of the alkenyl, contains at least one —C≡C-bond and in the case of the alkinyl, contains at least one —C≡-C-bond;
-alkyl, -alkenyl, resp., -alkinyl are linear or branched, -cycloalkyl and
-cycloalkenyl represent a group with 3 up to 20 carbon atoms; the heterocyclic groups represent a residue with 1 up to 20 carbon atoms, wherein up to 5 carbon atoms have been substituted by heteroatoms selected from the following: nitrogen, oxygen, sulphur, phosphorus; -aryl represents an aromatic residue with 5 up to 20 carbon atoms; and heteroaryl represents a corresponding aromatic residue wherein up to 5 carbon atoms have been substituted by heteroatoms selected from the following: nitrogen, oxygen, sulphur, phosphorus,
wherein L optionally carries 1 up to 3 substituents selected from the following: F; Cl; Br; I; —OH; —O—(C₁-C₁₀-alkyl); —SH; —S—(C₁-C₁₀-alkyl); —SO₃H; —CN; —COOH; —COO—(C₁-C₁₀-alkyl); —O—(C═O)——(C₁-C₁₀-alkyl); —CONH₂; —CONH(C₁-C₁₀-alkyl); —CON(C₁-C₁₀-alkyl)₂, wherein the two alkyl groups are identical or different; —NH₂, —NH(C₁-C₁₀-alkyl); —N(C₁-C₁₀-alkyl)₂, wherein the two alkyl groups are identical or different; and wherein alkyl groups are linear or branched,
and/or R1 and R2 represent, independently from each other,
—OH, —O⁻, —OL, —SH, —S—, —SL, —SOH, —SO₂H, —SO₃H, —(S═O)-L, —SO₂L, —NO, —NO₂, —C≡N, —C═N-L, —N≡C, —N═C-L, —NH₂, —NHL, —NH₂L⁺, —NYZ, —NHYZ⁺, —CHO, —COL, —COOH, —COO⁻, —COOL, —O(CHO), —O(C═O)L, —CONH₂, —CONHL, —CONYZ, —NHCOOH, —NLCOOH, —NLCOOL, —NHCOOL, —NH—(C═O)L, —NH—(C═N—H)—NH₂, —NH—(C═N—H)—NYZ, —NY—(C═N—Z)—NHL, —NH—(C═N—H)—NHL, —SO₂—NH-L, —SO₂—NH₂, —SO₂—NYZ, —NY—SO₂Z, —O—(CₚH₂ₚ)ₓ—13 O-L; —O—(CₚH₂ₚ)ₓ—O-L; —O—(CₚH₂ₚ—O)ₓ-L; —(CₚH₂ₚ—O)ₓ-L; wherein p represents a natural number from 1 up to 4 and x represents a natural number from 1 up to 10, and
wherein Y and Z, independently from each other, have the meanings described for L,
and wherein terminal amino groups are optionally present in the form of their hydrohalides, acetamides, mono-, di- or trihaloacetamides, wherein “halo-” resp. “halide” means fluorine and/or chlorine and/or bromine and/or iodine,
and/or wherein R1 and R2 represent, independently from each other, a fatty acid residue
—CH₂—(CᵣH₂ᵣ)—COOH, —CH₂—(CᵣH₂ᵣ₋₂)—COOH, —CH₂—(CᵣH₂ᵣ₋₄)—COOH,
—CH₂—(CᵣH₂ᵣ₋₆)—COOH, —CH₂—(CᵣH₂ᵣ₋₈)—COOH or an adamantane-1-yl-ester of one of these fatty acid residues and
r represents a natural number from 10 up to 18,
and/or wherein R1 and R2 represent, independently from each other, the residue R6 of an amino acid

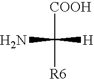

wherein R6 is preferentially benzyl-, 4-hydroxy-benzyl-, -(1H-indolyl)-methyl-, (1H-imidazolyl)-methyl-, 4-amino-butyl-, (3-guanidyl)-propyl, (2-methylthio)-ethyl, hydroxymethyl-, (R)-(1-hydroxy)-ethyl, (S)-(1-hydroxy)-ethyl, (2-carboxy)-ethyl-, (R)-(2-carbamoyl- 1-methyl)-ethyl, (S)-(2-carbamoyl-1-methyl)-ethyl-, carboxymethyl-, thiomethyl-, (2-carbamoyl)-ethyl-, (carbamoyl)-methyl-, selenomethyl-, (3-amino)-propyl-, 2-aminophenyl-2-oxo-ethyl-.
R3=—H or L, wherein L has the meanings listed under R1, R2 and wherein L optionally carries one up to three substituents selected from the following: —F; —Cl; —Br; —I; —OH; —O—(C₁-C₁₀-alkyl); —SH; —S—(C₁-C₁₀-alkyl); —SO₃H; —CN; —COOH; —COO—(C₁-C₁₀-alkyl); —O—(C═O)—(C₁-C₁₀-alkyl); —(C₁-C₁₀-alkyl); —CONH₂; —CONH(C₁-C₁₀-alkyl); —CON(C₁-C₁₀-alkyl)₂, wherein the two alkyl groups are identical or different; —NH₂, —NH(C₁-C₁₀-alkyl); —N(C₁-C₁₀-alkyl)₂, wherein the two alkyl groups are identical or different; and wherein the alkyl groups are linear or branched,
A=represents a single bond or
—(C═O)-(AA)ₘ-, wherein each unit of AA represents a natural or non-natural α-, β-, γ- or δ- amino acid in the D or L configuration;
m represents an integer from 0 up to 10,
the amino acids within the peptide chain m are identical or different, and wherein the N terminus of (AA)ₘ is bound to the C═O-group listed in the definition of A and the C terminus of (AA)ₘ is bound to R4,
or A represents a 2- up to 10-membered depsipeptide, wherein each amino acid within the depsipeptide represents a natural or non-natural α-, β-, γ- or δ- amino acid in the D or L configuration and wherein each carboxylic acid within the depsipeptide represents an aliphatic, aromatic, or araliphatic carboxylic acid with 1 up to 10 carbon atoms, wherein aliphatic groups are linear, cyclic, or branched, and wherein each carboxylic acid optionally carries one up to three substituents selected from the following:
—OH, —(C═O)—, —COOH, —F, —Cl,
—Br, —I, —NH₂, —SH, —S—S—,
   R4=—H or L, wherein L has the meanings listed under R1, R2 and wherein L optionally carries one up to three substituents selected from the following: —F; —Cl; and contains —Br; —I; —OH; —O—(C₁-C₁₀-alkyl); —SH; —S—(C₁-C₁₀-alkyl); —SO₃H; —CN; —COOH; —COO—(C₁-C₁₀-alkyl); —O—(C═O)—(C₁-C₁₀-alkyl); —(C₁-C₁₀-alkyl); —CONH₂; —CONH(C₁-C₁₀-alkyl); —CON(C₁-C₁₀-alkyl)₂, wherein the two alkyl groups are identical or different; —NH₂, —NH(C₁-C₁₀-alkyl); —N(C₁-C₁₀-alkyl)₂, wherein the two alkyl groups are identical or different; and wherein the alkyl groups are linear or branched, 1-adamantyl optionally containing one up to three substituents selected from the following: —F; —Cl; —Br; —I; —OH; —O—(C₁-C₁₀-alkyl); —SH; —S—(C₁-C₁₀-alkyl); —SO₃H; —CN; —COOH; —COO—(C₁-C₁₀-alkyl); —CONH₂; —CONH(C₁-C₁₀-alkyl); —CON(C₁-C₁₀-alkyl)₂, wherein the two alkyl groups are identical or different; —NH₂, —NH(C₁-C₁₀-alkyl); —N(C₁-C₁₀-alkyl)₂, wherein the two alkyl groups are identical or different, and each of the alkyl groups is linear or branched and has the meanings listed under R1 and R2,
   when A=—(C=O)-(AA)ₘ-
   or
   R4 has the meanings listed under R1 and R2 if A=single bond,
   B=single bond,
or (AA)q, wherein each unit of the amino acid represents a natural or non-natural α-, β-, γ- or δ- amino acid in the D or L configuration,
q represents an integer form 0 up to 10,
the amino acids within the peptide chain q are identical or different and wherein the C terminus of (AA)_{q} is bound to the nitrogen atom which is bound to the adamantyl residue and the N terminus of (AA)_{q} is bound to R5;
or B represents a 2- up to 10-membered depsipeptide, in which each amino acid within the depsipeptide represents a natural or non-natural α-, β-, γ- or δ- amino acid in the D or L configuration and each carboxylic acid within the depsipeptide represents an aliphatic, aromatic or araliphatic carboxylic acid with 1 up to 10 carbon atoms, wherein aliphatic groups are linear, cyclic or branched and each carboxylic acid optionally carries one up to three substituents selected from the following: —OH, —(C═O)—, —COOH, —F, —Cl, 'Br, —I, —NH₂, —SH, —S—S—,
   R5=—H or L, wherein L has the meanings listed under R1, R2; and L optionally carries one up to three substituents selected from the following: —F; —Cl; —Br; —I; —H; —O—(C₁-C₁₀-alkyl); —SH; —S—(C₁-C₁₀-alkyl); —SO₃H; —CN; —COOH;
   contains —COO—(C₁-C₁₀-alkyl); —O—(C═O)—(C₁-C₁₀-alkyl); —(C₁-C₁₀-alkyl); —CONH₂; —CONH(C₁-C₁₀-alkyl); —CON(C₁-C₁₀-alkyl)₂, wherein the two alkyl groups are identical or different; —NH₂, —NH(C₁-C₁₀-alkyl); —N(C₁-C₁₀-alkyl)₂, wherein the two alkyl groups are identical or different; and wherein the alkyl groups are linear or branched;
   or
   —SO₂L, —(C═O)-L; —COOL; —(CₚH₂ₚ)ₓ—O-L; —(CₚH₂ₚ—O)ₓ-L; wherein p, x and L have the meanings listed under R1 and R2, or
   wherein 1-adamantyl optionally contains one up to three substituents selected from the following: —F; —Cl; —Br; —I; —OH; —O—(C₁-C₁₀-alkyl); —SH; —S—(C₁-C₁₀-alkyl); —SO₃H; —CN; —COOH; —COO—(C₁-C₁₀-alkyl); —CONH₂; —CONH(C₁-C₁₀-alkyl); —CON(C₁-C₁₀-alkyl)₂, in which the two alkyl groups are identical or different; —NH₂, —NH(C₁-C₁₀-alkyl); —N(C₁-C₁₀-alkyl)2, wherein the two alkyl groups are identical or different, and the alkyl groups have the meanings listed under R1 and R2,
   and
   n=1-40 and a natural number, if this is an open-chained compound in which R1, R2, R3, A and B are identical or different in every unit n and in which at least one of the groups R1, R2 and R4 is different from hydrogen,
   or
   R4, R5=single bonds and n=1-25 in the case of a cyclic oligopeptide, wherein R1, R2, R3, A and B are identical or different in every unit, wherein B represents a sequence (AA)_{z} of natural or non-natural α-, β-, γ- or δ- amino acids in the D or L configuration or a 1 to 25-membered depsipeptide, every amino acid within the depsipeptide represents a natural or non-natural α-, β-, γ- or δ- amino acid in the D or L configuration and every carboxylic acid within the depsipeptide represents an aliphatic, aromatic or araliphatic carboxylic acid with 1 up to 10 carbon atoms; wherein aliphatic groups are linear, cyclic or branched, and every carboxylic acid optionally carries one up to three substituents selected from the following: —OH, —(C═O)—, 'COOH, —F, —Cl, —Br, —I, —NH₂, —SH, —S—S—,
   and z, in the case of n=1, is a natural number between 3 and 25, or
   z, in the case of n=2, is a natural number between 2 and 25, or
   B, in the case of n=1, is a 3-aminoadamantane-carboxylic acid based on the present invention,
   and with the proviso that n=1 and both A and B are single bonds, such compounds are excluded wherein, according to the definition of the general structural formula above (cf. p. 15), the following combinations of residues of R1, R2, R3, R4 and R5 are represented:
   compounds in which R1, R2 and R3 are hydrogen, R4 is a carboxyl group and R5 is hydrogen or an acetyl group,
   a compound in which R1, R2 and R3 are hydrogen, R4 is a carboxylmethyl group and R5 is an acetyl group,
   a compound in which R1 and R2 are methyl groups and R3, R4 and R5 are hydrogen,
   as well as compounds in which R1 and R2 have been selected, independently from each other, from —H, —F, —Cl, —Br, —I, —CN, —COOH, 'CH₃, 'CH₂CH₃, —OCH₃, —OCH₂CH₃, —CH₂CH₂CH₃, —CH(CH₃)₂, —CH₃—O—(C=O)—, CH₃—O—(C═O)—and CH₃CH₂—O—(C═O)—; R3 is hydrogen and R4 is a carboxyl group; and R5 has been selected from the following:
   3-formylamino-2-hydroxy-benzoyl-, 3-acetylamino-2-hydroxy-benzoyl-, 2-hydroxy-3-propionylamino-benzoyl-, 2-butyrylamino-2-hydroxy-benzoyl-, 2-hydroxy-3-isobutyrylamino-, 2-hydroxy-3-pentanoylamino-benzoyl-, 2-hydroxy-3-(3-methyl-butyrylamino-)benzoyl-, 2-hydroxy-3-(2-methyl-butyrylamino)-benzoyl-, 3-(2,2-dimethyl-propionylamino)-2-hydroxy-benzoyl-, 2-hydroxy-3-methoxycarbonylamino-benzoyl-, 2-hydroxy-3-ethoxycarbonylamino-benzoyl-, 2-hydroxy-3-propoxycarbonylamino-benzoyl-, 2-hydroxy-3-isopropoxycarbonylamino-benzoyl-,
   furthermore, such compounds are excluded wherein A and B are single bonds, n=1, R3 and R5 have been selected, independently from each other, from H, aryl, or heteroaryl with up to 7 ring elements, C₁-C₁₀-alkyl, C₁-C₁₀-alkenyl and C₁-C₁₀-alkinyl, wherein the alkyl, alkenyl and alkinyl residues are branched, linear or cyclic and may have been substituted with halogen, aryl or heteroaryl with up to 7 ring elements,
   and R1, R2 and R4 have been selected, independently from each other, from —NR19R20, H, aryl or heteroaryl with up to 7 ring elements, C₁-C₁₀-alkyl, C₁-C₁₀-alkenyl and C₁-C₁₀-alkinyl, wherein the alkyl-, alkenyl- and alkinyl residues are branched, linear or cyclic and may have been substituted with halogen, aryl or heteroaryl with up to 7 ring elements, and R19 and R20 have been selected, independently from each other, from H, aryl or heteroaryl with up to 7 ring elements, C₁-C₁₀-alkyl, C₁-C₁₀-alkenyl and C₁-C₁₀-alkinyl, wherein the alkyl-, alkenyl- and alkinyl residues are branched, linear or cyclic and may have been substituted with halogen, aryl or heteroaryl with up to 7 ring elements; or R19 and R20, together with the nitrogen atom, form a heterocyclic group with up to 7 ring elements,
   furthermore, such compounds are excluded wherein A and B are single bonds, n=1, residues R1 and R2 represent lower straight-chained or branched alkyl residues, R3 represents hydrogen, a lower straight-chained, branched or cyclic alkyl residue and the residue R5 represents hydrogen or the methyl group.

The compounds based on the present invention are produced by means of the methods based on the invention as illustrated below:

### Method for the Production of 3,5-Substituted 1-Aminoadamantane Carboxylic Acid Amides via Direct Acetamidation of 1,3-Disubstituted Adamantane Derivatives)

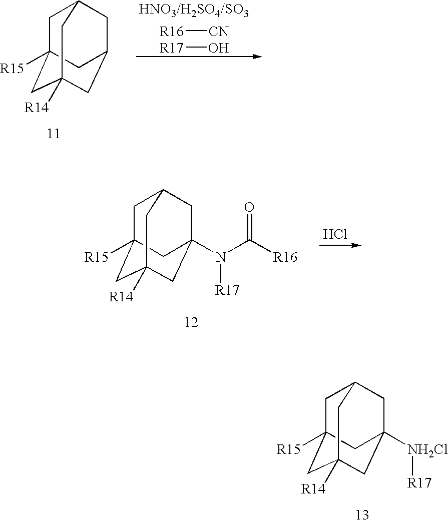

wherein R14 and R15 are independent from each other:

### H;

L=-alkyl, -cycloalkyl, -heterocycloalkyl, -aryl, -heteroaryl, -alkylaryl, -alkylheteroaryl, -alkylcycloalkyl, -alkylheterocycloalkyl,
wherein -alkyl represents a group with 1 up to 10 carbon atoms, preferentially methyl, ethyl, propyl, isopropyl, 1-butyl, 2-butyl, (2-methyl-)propyl, tert.-butyl; -alkyl can be linear or branched, -cycloalkyl represents a group with 3 up to 20 carbon atoms; the heterocyclic groups represent a residue with 1 up to 20 carbon atoms, wherein up to 5 carbon atoms are substituted by heteroatoms selected from the following: nitrogen, oxygen, sulphur, phosphorus, -aryl represents an aromatic residue with 5 up to 20 carbon atoms and heteroaryl represents a corresponding aromatic residue wherein up to 5 carbon atoms have been substituted by heteroatoms selected from the following: nitrogen, oxygen, sulphur, phosphorus,
wherein L optionally contains one up to three substituents selected from the following: —O—(C₁-C₁₀-alkyl); —S—(C₁-C₁₀-alkyl); —SO₃H; —COOH; —COO—(C₁-C₁₀-alkyl); —O—(C═O)——(C₁-C₁₀-alkyl); —NH₂, —NH(C₁-C₁₀-alkyl); —N(C₁-C₁₀-alkyl)₂, wherein the two alkyl groups are identical or different, and wherein alkyl groups are linear or branched.
and/or R14 and R15 are independent from each other
—OL, —SL, —SO₃H, —SO₂L, —NO₂, —NH₂, —NHL, —NH₂L⁺, —NYZ, —NHYZ⁺, —COL, —COOH, —COO—, —COOL, —NLCOOH, —NLCOOL, —NHCOOL, —NH—(C═N—H)—NH₂, —NH—(C═N—H)—NYZ, —NY—(C═N-Z)—NHL, —NH—(C═N—H)—NHL, —SO₂—NH-L, —SO₂—NH₂, —SO₂—NYZ, —Y—SO₂Z, —O—(CₚH₂ₚ)ₓ—O-L; —(CₚH₂ₚ)ₓ—O-L; —O—(CₚH₂ₚ—O)ₓ-L; —(CₚH₂ₚ—O)ₓ-L; in which p can be a natural number from 1 up to 4, and x can be a natural number from 1 up to 10, and
wherein Y and Z, independently from each other, have the meanings described for L,
and in which terminal amino groups are optionally present in the form of their hydrohalides,
and/or wherein R14 and R15, independently from each other, represent a fatty acid residue
—CH₂—(CᵣH₂ᵣ)—COOH, —CH₂—(CᵣH₂ᵣ₋₂)—COOH; —CH₂—(CᵣH₂ᵣ₋₄)—COOH,
—CH₂—(CᵣH₂ᵣ₋₆)—COOH, —CH₂—(CᵣH₂ᵣ₋₈)—COOH or an adamantane-1-yl-ester of one of these fatty acid residues and
r is a natural number from 10 up to 18;
wherein R16 represents
-alkyl, -cycloalkyl, -heterocycloalkyl, -aryl, -heteroaryl, -alkylaryl, -alkylheteroaryl, -alkylcycloalkyl, -alkylheterocycloalkyl,
wherein -alkyl represents a group with 1 up to 10 carbon atoms, preferentially methyl, ethyl, propyl, isopropyl, 1-butyl, 2-butyl, (2-methyl-)propyl, tert.-butyl, 1-adamantyl and -alkyl are linear or branched, -cycloalkyl and the heterocyclic groups represent a residue with 1 up to 20 carbon atoms, wherein up to 5 carbon atoms have been substituted by heteroatoms selected from the following:
nitrogen, oxygen, sulphur, phosphorus; -aryl represents an aromatic residue with 5 up to 20 carbon atoms, and heteroaryl represents a corresponding aromatic residue wherein up to 5 carbon atoms have been substituted by heteroatoms selected from the following: nitrogen, oxygen, sulphur, phosphorus,
wherein L optionally contains one up to three substituents selected from the following: —O—(C₁-C₁₀-alkyl); —S—(C₁-C₁₀-alkyl); —SO₃H; —COOH; —NH₂, —NH(C₁-C₁₀-alkyl); —N(C₁-C₁₀-alkyl)₂, wherein the two alkyl groups are identical or different; and wherein alkyl groups are linear, cyclic or branched,
and
wherein R17 represents
—H, -alkyl, -cycloalkyl, -heterocycloalkyl, -aryl, -heteroaryl, -alkylaryl, -alkylheteroaryl, -alkylcycloalkyl, -alkylheterocycloalkyl,
in which -alkyl represents a group with 1 up to 10 carbon atoms, preferentially for methyl, ethyl, propyl, isopropyl, 1-butyl, 2-butyl, (2-methyl-)propyl, tert.-butyl, 1-adamantyl and -alkyl are linear or branched, -cycloalkyl and the heterocyclic groups represent a residue with 1 up to 20 carbon atoms, wherein up to 5 carbon atoms have been substituted by heteroatoms selected from the following:
nitrogen, oxygen, sulphur, phosphorus; -aryl represents an aromatic residue with 5 up to 20 carbon atoms, and heteroaryl represents a corresponding aromatic residue in which 5 carbon atoms have been substituted by heteroatoms selected from the following: nitrogen, oxygen, sulphur, phosphorus,
wherein L optionally contains one up to three substituents selected from the following: —O—(C₁-C₁₀-alkyl); —S—(C₁-C₁₀-alkyl); —SO₃H; —COOH; —NH₂, —NH(C₁-C₁₀-alkyl); —N(C₁-C₁₀-alkyl)₂, wherein the two alkyl groups are identical or different, and wherein alkyl groups are linear, cyclic, or branched.

Based on the method for the direct introduction of carboxylic acid amides according to the present invention, 1 equivalent of 1,3-dimethyladamantane (11, R14=R15=CH₃) is suspended in a mixture of conc. HNO₃ and conc. H₂SO₄ (95-98%) (e.g. 5:6 by volume) at 10-30 ° C., preferentially at room temperature. Hereby, 1 equivalent is equal to a multiple of 0.9 mol to 1.1 mol and one part is equal to the same multiple of 0.2 L to 0.3 L. To this mixture, five parts oleum (20-40%, preferentially 30% SO₃) are added at −15° C. to +15° C., preferentially at 0° C., and the resulting clear solution is continually stirred for a further 0.5-53 h at −10 to +5° C., preferentially 1 h at 0° C., and for a further 2-4 h, preferentially 3 h, at 15-25° C., preferentially 20° C.

Alternatively, concentrated, preferentially 100%, HNO₃ can be utilised without the further addition of H₂SO₄ or oleum. Hereby, the 1,3-dimethyladamantane is suspended or dissolved in 100% HNO₃, producing, preferentially, a solution. Subsequently, it is cooled down to an inside temperature of −10° C. to +10° C., preferentially 0° C., and within 10-20 minutes, preferentially 15 minutes, 3-6, preferentially 4, parts nitrile are added. After completing this addition, continual stirring occurs for 5-15 minutes at −5° C. to +5° C., preferentially 10 minutes at 0° C., and for 2-4 h at 20-30° C., preferentially for 3 h at room temperature. Subsequently, the reaction mixture is poured onto 300-600 g of ice if, in the product produced, R17=H. Alternatively, a mixture of an alcohol R17-OH and diethylether is added to the reaction mixture (volume: 1:1), if, in the product produced, R17 is not hydrogen, with the volume of the alcohol/ether mixture essentially corresponding to the volume of the reaction mixture.

The resulting solution is extracted 2 up to 5 times with 4 parts diethylether each time. The combined organic phases are dried with anhydrous Na₂SO₄ and filtered through 5-10 parts basic metal oxide, preferentially basic Al₂O₃. Alternatively, methylene chloride, ethyl acetate, or chloroform and/or further unpolar organic solvents can be used for the extraction; alternatively to Na₂SO₄, other common desiccants such as anhydrous MgSO₄ can also be used for drying the crude product. After concentrating the solvent at reduced pressure, preferentially at 15 up to 60 mbar, and a temperature of 40° C. up to 90° C., preferentially 60° C., the amide is obtained in the form of a white solid yielding 60% up to 98%.
The alcohol R17-OH is, preferentially methanol, ethanol, n-propanol, n-butanol. The ether utilised for the production of the alcohol-ether-mixture is a dialkylether, preferentially diethylether, diisopropylether or tert.-butylmethylether.

Optionally, via reaction with a concentrated mineral acid, e.g. HCl, the amide 12 can subsequently react to form the salt of the corresponding amine. If HCl is used, the hydrochloride of the amine is thereby obtained. This conversion of amides to the corresponding amines is known to the person skilled in the art and can be consulted, for example, in: K. P. C. Vollhardt: “Organische Chemie”, VCH Verlagsgesellschaft, Weinheim, 1. ed. 1988.

It is easily apparent to the person skilled in the art that the production method according to the present invention described here is also suitable for the production of other 3,5-substituted and unsubstituted carboxylic acid amides of the adamantane.

In the case of R14, R15=methyl and R17=H, the method described here results in a reaction product 10 which is known to the person skilled in the art under the name Memantine® and available in trade.
By starting with unsubstituted adamantane (R14=R15=H), one obtains—after the cleavage of the acetamide which was first formed with mineral acids—the 1-aminoadamantane hydrochloride which is known to the person skilled in the art as an antiviral agent, among other things, and which is commercially available under the name Amantadine®.

### Method for the Production of 5,7-Disubstituted 3-Aminoadamantane-1-Carboxylic Acids

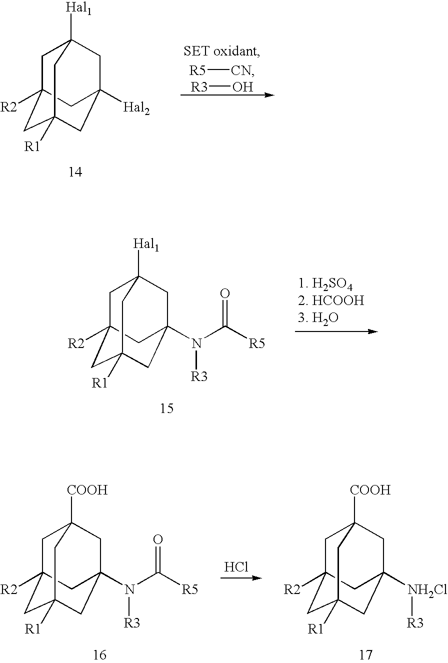

Based on the method for the production of 5,7-disubstituted 3-aminoadamantane-1-carboxylic acids according to the present invention, mixed dihalogenoadamantane derivatives 14 react selectively into monohalocarboxylic acid amides 15. Here, mixed dihalides are considered to be 1,3-dihalogenoadamantane derivatives in which the two halogen atoms are different. Here, selective is considered to mean that the more reactive one of the two halogen atoms is converted into a carboxylic acid amide, wherein an order of reactivity of halogens in the order I>Br>Cl>F applies. Such a selective reaction of mixed dihalides is not known in the current technical state of the art.

Optionally, the halogeno carboxylic acid amides 15 produced according to the method based on the present invention can subsequently be converted into acetamidoadamantane carboxylic acid derivatives 16 with conc. sulphuric acid and CO produced in situ, after an aqueous workup. The cleavage of the acetamide occurs by heating the carboxylic acid amides with conc. HCl for 15 h up to 72 h. Thereby, the 5- and/or 7-substituted 3-aminoadamantane-1-carboxylic acids 17 are obtained in the form of hydrochlorides.

The mixed dihalides 14 react into monohalogeno carboxylic acid amide, according to the present invention. In order to achieve this, a 0.2 up to 0.6 molar solution of 1 up to 1.2 equivalents of a single electron oxidant, referred to as an “SET oxidant” in the synthesis scheme, is produced in a nitrile and cooled to −60° C. to −20° C., preferentially −50° C. At this temperature, a 0.04-0.3 molar solution of an equivalent of the mixed dihalogenoadamantane is added in the same nitrile within 20 minutes to 2 hours. The reaction mixture is stirred for 0.5-3 h, preferentially for 1 h, during which it warms to −15° C. to 0° C., preferentially −10° C. Subsequently, a mixture of water (or an alcohol) and diethylether is added to the reaction mixture at a volume ratio of 1:1, the volume of the water (resp. alcohol) / ether mixture corresponding, essentially, to the volume of the reaction mixture. After the phase separation, the aqueous phase is extracted two up to four times, preferentially three times, with 0.2 parts ether and the combined etheric phases are subsequently washed two up to four times, preferentially three times, with 0.2 parts saturated NaHSO₃ solution, then one to up three times, preferentially two times, with 0.2 parts water, and finally, one up to two times with 0.2 parts saturated saline solution. After drying, the solvents are removed at reduced pressure via destillation. Column chromatography over SiO₂ (column bed) and, for instance, diethylether or chloroform as the eluent yield the desired halocarboxylic acid amides in a yield of typically 80% to 95%.

The SET oxidant is preferentially NOBF₄ or NOSF₆.

In the 1,3-disubstituted adamantane derivates employed as educts, are R1 and R2=H, F, Cl, Br, I; or

L=-alkyl, -alkenyl, -alkinyl, -cycloalkyl, -cycloalkenyl, -heterocycloalkyl, -heterocycloalkenyl, -aryl, -heteroaryl, -alkylaryl, -alkylheteroaryl, -alkylcycloalkyl, -alkylheterocycloalkyl, -alkenylcycloalkyl, -alkenylheterocycloalkyl,
wherein -alkyl represents a group with 1 up to 10 carbon atoms, preferentially methyl, ethyl, propyl, isopropyl, 1-butyl, 2-butyl, (2-methyl-)propyl, tert.-butyl and -alkenyl and -alkinyl represent a monounsaturated or polyunsaturated group with 2 up to 10 carbon atoms which, in the case of the alkenyl, contains at least one —C═C-bond and in the case of the alkinyl, contains at least one —C≡C-bond; -alkyl, -alkenyl, resp., -alkinyl are linear or branched, -cycloalkyl and -cycloalkenyl represent a group with 3 up to 20 carbon atoms; the heterocyclic groups represent a residue with 1 up to 20 carbon atoms, wherein up to 5 carbon atoms have been substituted by heteroatoms selected from the following:
nitrogen, oxygen, sulphur, phosphorus; -aryl represents an aromatic residue with 5 up to 20 carbon atoms; and heteroaryl represents a corresponding aromatic residue wherein up to 5 carbon atoms have been substituted by heteroatoms selected from the following: nitrogen, oxygen, sulphur, phosphorus, wherein L optionally carries 1 up to 3 substituents selected from the following: F; Cl; Br; I; —OH; —O—(C₁-C₁₀-alkyl); —SH; —S—(C₁-C₁₀-alkyl); —SO₃H; —CN; —COOH; —COO—(C₁C₁₀-alkyl); —O—(C═O)——(C₁-C₁₀-alkyl); —CONH₂; —CONH(C₁-C₁₀-alkyl); —CON(C₁-C₁₀-alkyl)₂, wherein the two alkyl groups are identical or different; —NH₂, —H(C₁-C₁₀-alkyl); —N(C₁-C₁₀-alkyl)₂, wherein the two alkyl groups are identical or different; and wherein alkyl groups are linear or branched,
and/or R1 and R2 represent, independently from each other,
—OH, —O—, —OL, —SH, —S—, —SL, —SOH, —SO₂H, —SO₃H, —(S═O)-L, —SO₂L, —NO, —NO₂, —C≡N, —C═N-L, —N≡C, —N=C-L, —NH₂, —NHL, —NH₂L⁺, —NYZ, —NHYZ⁺, —CHO,
—COL, —COOH, —COO—, —COOL, —O(CHO), —O(C═O)L, —CONH₂, —CONHL, —CONYZ, —NHCOOH, —NLCOOH, —NLCOOL, —NHCOOL, —NH—(C═O)L, —NH—(C═N—H)—NH₂, —NH—(C═N—H)—NYZ, —NY—(C═N-Z)—NHL, —NH—(C═N—H)—NHL, —SO₂—NH-L, —SO₂—NH₂, —SO₂—NYZ, —NY—SO₂Z, —O—(CₚH₂ₚ)ₓ—O-L; —(CₚH₂ₚ)ₓ—O-L; —O—(CₚH₂ₚ—O)ₓ-L; —(CₚH₂ₚ—O)ₓ-L; wherein p represents a natural number from 1 up to 4 and x represents a natural number from 1 up to 10, and
wherein Y and Z, independently from each other, have the meanings described for L,
and wherein terminal amino groups are optionally present in the form of their hydrohalides, acetamides, mono-, di- or trihaloacetamides, wherein “halo-” resp. “halide” means fluorine and/or chlorine and/or bromine and/or iodine,
and/or wherein R1 and R2, independently from each other, represent a fatty acid residue
—CH₂—(CᵣH₂ᵣ)—COOH, —CH₂—(CᵣH₂ᵣ₋₂)—COOH, —CH₂—(CᵣH₂ᵣ₋₄)—COOH,
—CH₂—(CᵣH₂ᵣ₋₆)—COOH, —CH₂—(CᵣH₂ᵣ₋₈)—COOH or an adamantane-1-yl-ester of one of these fatty acid residues and
r represents a natural number from 10 up to 18,
and/or wherein R1 and R2, independently from each other, represent the residue R6 of an amino acid,

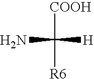

wherein R6 preferentially represents benzyl-, 4-hydroxy-benzyl-, -(1 H-indolyl)-methyl-, (1H-imidazolyl)-methyl-, 4-amino-butyl-, (3-guanidyl)-propyl, (2-methylthio)-ethyl, hydroxymethyl-, (R)-(1-hydroxy)-ethyl, (S)-(1-hydroxy)-ethyl, (2-carboxy)-ethyl-, (R)-(2-carbamoyl-1-methyl)-ethyl, (S)-(2-carbamoyl- 1-methyl)-ethyl-, carboxymethyl-, thiomethyl-, (2-carbamoyl)-ethyl-, (carbamoyl)-methyl-, selenomethyl-, (3-amino)-propyl-, 2-aminophenyl-2-oxo-ethyl-.
R5 represents —H or L, wherein L has the meanings listed under R1, R2; and L optionally carries one up to three substituents selected from the following: —F; —Cl; —Br; —I; —OH; —O—(C₁-C₁₀-alkyl); —SH; —S—(C₁-C₁₀-alkyl); —SO₃H; —CN; —COOH; —COO—(C₁-C₁₀-alkyl); —O—(C═O)—(C₁-C₁₀-alkyl); —(C₁-C₁₀-alkyl); —CONH₂; —CONH(C₁-C₁₀-alkyl); —CON(C₁-C₁₀-alkyl)₂, wherein the two alkyl groups are identical or different; contains —NH₂, —NH(C₁-C₁₀-alkyl); —N(C₁-C₁₀-alkyl)₂, wherein the two alkyl groups are identical or different; and wherein the alkyl groups are linear or branched;
or
—SO₂L, —(C═O)-L; —COOL; —(CₚH₂ₚ)ₓ—O-L; —(CₚH₂ₚ—O)ₓ-L; wherein p, x and L have the meanings listed under R1 and R2, or
1-adamantyle optionally containing one up to three substituents selected from the following: —F; —Cl; —Br; —I; —OH; —O—(C₁-C₁₀-alkyl); —SH; —S—(C₁-C₁₀-alkyl); —SO₃H; —CN; —COOH; —COO—(C₁-C₁₀-alkyl); —CONH₂; —CONH(C₁-C₁₀-alkyl); —CON(C₁-C₁₀-alkyl)₂, wherein the two alkyl groups are identical or different; —NH₂, —NH(C₁-C₁₀-alkyl); —N(C₁-C₁₀-alkyl)2, wherein the two alkyl groups are identical or different and the alkyl groups have the meanings listed under R1 and R2. Preferentially, R5-CN represents acetonitrile, chloroacetonitrile, trichloroacetonitrile, propionitrile, chloropropionitrile, n-butyronitrile, 3-carboxyadamantane-1-carbonitrile.

R3 represents —H or L, wherein L has the meanings listed under R1, R2 and L optionally carries one up to three substituents selected from the following: —F; —Cl; —Br; —I; —OH; —O—(C₁-C₁₀-alkyl); —SH; —S—(C₁-C₁₀-alkyl); —SO₃H; —CN; —COOH; —COO—(C₁-C₁₀-alkyl); —O—(C═O)—(C₁-C₁₀-alkyl); —(C₁-C₁₀-alkyl); —CONH₂; —CONH(C₁—C₁₀-alkyl); —CON(C₁-C₁₀-alkyl)₂, wherein the two alkyl groups are identical or different; contains —NH₂, —NH(C₁-C₁₀-alkyl); —N(C₁-C₁₀-alkyl)₂, wherein the two alkyl groups are identical or different; and wherein the alkyl groups are linear or branched. Preferentially, the alcohol R3-OH utilised for the production of the alcohol-ether mixture represents methanol, ethanol, n-propanol, n-butanol. The ether utilised for the production of the water- or alcohol-ether mixture represents a dialkylether, preferentially diethylether, diisopropylether or tert.-butyl-methylether. The production of amides from arylalkylhalides and nitriles in the presence of SET oxidants is known to the person skilled in the art and has been described in: G. A. Olah, B. G. Gupta, S. C. Narang: Synthesis-Stuttgart 1979, 274-276.

Surprisingly, this method has also been found to be suitable for the direct and selective introduction of carboxylic acids in mixed 1,3-dihalogenoadamantane derivates. Under the reaction conditions based on the present invention, mixed dihalides react in a chemoselective manner, i.e. the more reactive halogen abreacts completely before the less reactive halogen is affected, with their reactivity decreasing in the order I>Br>Cl>F.

Besides the selection of the reaction conditions during PTC and the introduction of the carboxylic acid amide, the selection of the equivalent concentration of SET oxidant is crucial.

The halogenoamides obtained via the method based on the present invention for the direct production of carboxylic acid amides from mixed 1,3-dihalogenoadamantane derivates are converted to the corresponding carboxylic acid in a Koch-Haaf reaction with carbon monoxide produced in situ. The Koch-Haaf reaction is known to the person skilled in the art and can be found in organic chemistry textbooks, e.g.: J. March: Advanced Organic Chemistry, Third Edition, John Wiley & Sons, New York, 1985. For this purpose, a solution of the haloacetamide in conc. sulphuric acid (95-98%) is produced and carbon monoxide is added to it for 2 up to 6 hours, preferentially 3 hours. The addition of carbon monoxide can be carried out in the manner known to the person skilled in the art, either by discharging as a CO gas or by the in situ production of CO, during which formic acid is added dropwise. Subsequently, the reaction mixture is poured onto ice; the crude product is filtered and recrystallised. Suitable solvents for the recrystallisation are methanol, acetic acid, formic acid, acetone, water, and mixtures thereof.

The cleavage of the amides is known to the person skilled in the art and can be carried out, for example, by heating in a concentrated mineral acid.

Alternative Method for the Production of 5-and 5,7-Substituted 3-Aminoadamantane-1-Carboxylic Acids Respectively

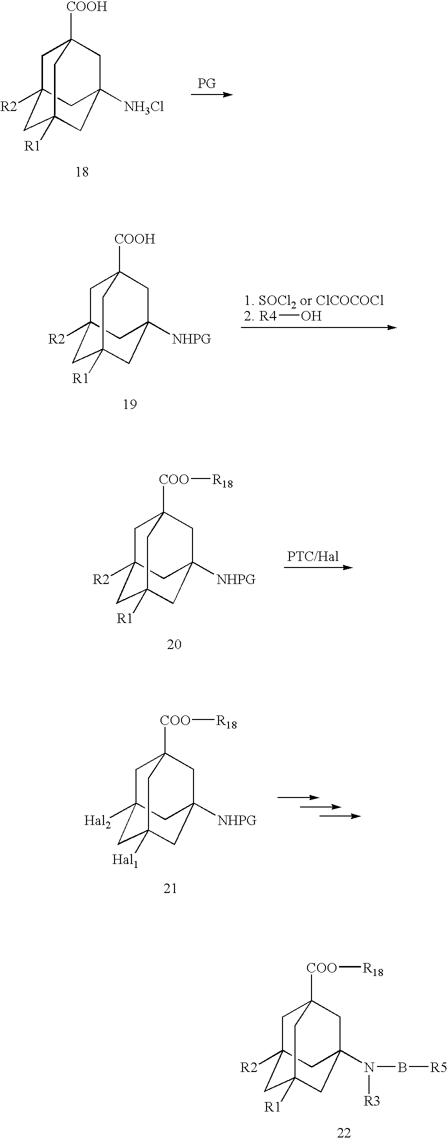

The introduction of further functional groups in the 5- and/or 7-position of the 3-aminoadamantane-1-carboxylic acids based on the present invention is possible by protecting them, in the case of R1 and/or R2=H, in a suitable way at the amino and carboxy group and subjecting them to renewed halogenation via phase transfer catalysis. The following selective reactions of the 5- and/or 7-halogen-substituted 3-aminoadamantane-1-carboxylic acid derivatives 21 obtained therein, in which hall and hal2 can be the same halogen or different halogens, with nucleophiles enable the introduction of a number of further substituents in 5- or 7-position. Particularly suitable nucleophiles for this are Grignard or organolithium reagents; alkali sulphites; and cyanide salts.

During the method for the introduction of further functional groups, based on the present invention, first a protective group, referred to in the scheme as “PG”, is introduced to the amino function of the 3-aminoadamantane-1-carboxylic acids 18 according to known protocols (T. W. Greene, P. G. M. Wuts, “Protective groups in organic synthesis”, 2^{nd} Edition 1991, John Wiley & Sons Inc., New York/Chichester/Brisbane/Toronto/Singapore). This protective group is selected from the following: acetal, acyl, silyl, benzyl protective groups, tert.-butyloxycarbonyl (Boc), benzyloxycarbonyl (Cbz), benzylether (Bn), and fluorenyl-9-methoxycarbonyl (Fmoc), preferentially Boc. Alternatively, the 3-amidoadamantane-1-carboxylic acids 16 are utilised. It is known to the person skilled in the art that the amido function as such already represents a protective group PG from which, by cleavage with mineral acids, the corresponding amines can be produced and optionally react into further amino derivatives.

The carboxyl function of the 3-acylamidoadamantane-1-carboxylic acids 16 or 3-aminoadamantane-1-carboxylic acids 18 based on the present invention are preferentially protected via esterification by first producing the corresponding carboxylic acid chloride with the help of thionyl chloride or oxalyl chloride and then reacting it with an alcohol R18-OH.
Here, R18=alkyl, with alkyl containing 1 up to 10 C-atoms and being linear or branched, and/or cycloalkyl with 3 up to 10 C-atoms and/or 1-adamantyl.
The substances 22 protected in this way at the carboxyl and amino group are subjected again to halogenation by phase transfer catalysis.

The further reaction of the halides 21 into the substances 22 based on the present invention occurs analogously to the method described in “Methods for the production of 3,5-disubstituted 3-aminoadamantane-1-carboxylic acids”. In this, the substances 22 are the monomeric compounds based on the present invention according to claim 1 (cf. general structural formula on p. 15).

### Enantiomer Separation of Chiral 3-Aminoadamantane-1-Carboxylic Acids

The separation of chiral 3-aminoadamantane-1-carboxylic acid derivatives based on the present invention (R1≠R2) is possible in two ways:
a.) Analytical Enantiomer Separation via Chiral HPLC.
   The 3-aminoadamantan-1-carboxylic acid derivative based on the present invention is converted analogously to an amide 19 if it does not already exist as an amide, and the carboxy group is converted to an ester 20. This chiral ester (R1≠R2) is separated analytically into the enantiomers via chiral HPLC. Chiral HPLC is known to the person skilled in the art and can be consulted, for instance, in: A. Henschen, K. P. Hupe, F. Lottspeich, W. Voelter (eds.), “High Performance Liquid Chromatography in Biochemistry”, VCH, Weinheim 1985, or in: G. Aced, H.J. Möckel, “Liquid-Chromatographie”, VCH, Weinheim 1991.
b.) Preparative Enantiomer Separation via Fractionated Cocrystallisation with Quinine
   In order to achieve this, analogously to a method known from literature (cf. Applequist, J., Rivers, P., Applequist, D. E. J. Am. Chem. Soc 1969, 91, 5705-5711), fractionated crystallisation of the respective acylaminoadamantane carboxylic acid 16 is carried out by adding 1 equivalent of quinine in a suitable solvent.” Suitable solvents are, for example, water, acetone, methanol, ethanol, 1-propanol, 2-propanol, dichloromethane, chloroform, hexane, toluene, nitromethane) or mixtures of these solvents. The free acid can be obtained by treating the salt with conc. hydrochloric acid at room temperature, adding water to the mixture, and extracting several times with an unpolar aprotic solvent, e.g. dichloromethane. The combined organic phases are washed with water and extracted with a 15-25% aqueous base, preferentially aqueous 20% NaOH. Alternatively, a 15-25% aqueous solution of LiOH, KOH, CsOH or Ba(OH)₂ can be utilised for this extraction as well. The alcalic extracts are acidified with a 20% up to 100% mineral acid, preferentially conc. HCl, under ice cooling, and the aminoadamantane carboxylic acid is sucked off, washed with water, and dried.

### Method for the Production of Oligomeric 3-Aminoadamantane-Carboxylic Acids

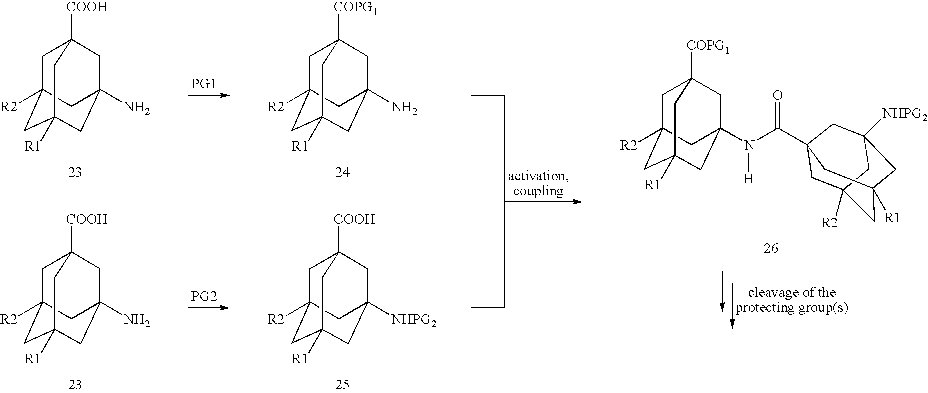

The protection of the amino-, and resp., carboxy function of the 3-aminoadamantane-1-carboxylic acid derivatives based on the present invention is carried out according to standard procedures which the person skilled in the art can consult in relevant literature, e.g. in: T. W. Greene and P. G. M. Wuts, “Protective groups in organic synthesis”, 2^{nd} Edition 1991, John Wiley & Sons Inc., New York / Chichester / Brisbane / Toronto / Singapore.

The 3-aminoadamantane-1-carboxylic acid derivatives based on the present invention 24, and resp., 25, which are thus protected, react into oligopeptides both in solution and in the solid phase (Solid Phase Peptide Synthesis, SPPS) following a suitable activation (cf. general structural formula on p. 15, n=240). Thereby, not only 3-aminoadamantane-1-carboxylic acid derivatives but also optional α-, β-, γ-, and δ-amino acids are coupled in a comparable manner.
The C-terminus of the peptide acid is activated by an activation reagent, which has been selected from the following: DIC, DCC, EDC, FmocOPfp, PyClop, HBTU, HATU, HOSu, TBTU, T3P, BopCl and 3-Cl-1-pyridiniumiodide. As coupling additives, the substances HOBt, HOAt and HONB known to the person skilled in the art can be utilised. It is known to the person skilled in the art that these reactions are functionally carried out in adding a base such as DIPEA. Further, several solvents are known to the person skilled in the art for utilisation in the methods listed. He can produce these combinations of activation reagents, coupling additives, bases, and solvents himself with his common knowledge and standard literature, e.g.: N. Sewald, H. D. Jakubke, “Peptides: Chemistry and Biology”, Weinheim, Wiley-VCH, 2002, and the works cited therein.

If the linear peptide contains further free COOH groups besides the C-terminal free COOH group within the peptide chain, e.g. COOH groups of glutamic acid and/or aspartic acid, these non-C-terminal free COOH groups must be protected from the reaction of the linear peptide with an activation reagent by means of an orthogonal protective group, which must be cleaved off again after producing the substrate based on the present invention. Suitable protective groups and suitable methods for their removal are known to the person skilled in the art and can be consulted, for instance, in: T. W. Greene and P. G. M. Wuts, Protective groups in organic synthesis”, 2^{nd} Edition 1991, John Wiley & Sons Inc., New York/Chichester/Brisbane/Toronto/Singapore.

The oligopeptides consisting of adamantane-amino acids display the following characteristic features:
resistance against peptide cleavers (acid, proteases); stability under a variety of conditions such as acid or basic conditions and high temperatures;
limited rotational degrees of freedom around the dihedron angles φ and ψ, which are limited in the so-called Ramachandran Plot, in comparison to native amino acids and the peptides formed thereof. The definition of φ and ψ is known to the person skilled in the art; he can find it in standard volumes such as: T. E. Creighton, “Proteins: Structure and molecular Properties”, 2^{nd} edition, W. H. Freeman, New York 1992. Due to the voluminous cycloaliphatic adamantane groups, an increased steric hindrance of different areas of φ and ψ, in comparison to native amino acids, occurs during rotation around the above mentioned dihedron angles φ and ψ. Steric hindrance here refers to the avoidance of interpenetration of the electron sheats of two molecules or moieties. It is known to the person skilled in the art that even during a generally minor interpenetration of the electron sheats of two molecules or moieties, each with a closed electron shell, antibinding interactions occur which result, in the end, in a destabilisation of the molecule configuration concerned. It is known that adamantane is considered to be a member of the group of aliphatic compounds with a so-called cage structure. “Cage” here refers to a closed, three-dimensional and polyhedral structure, with sp³-sp³-hybridised carbon atoms representing the corners of the polyhedron and carbon-carbon σ bonds representing the edges of the polyhedron. Due to its rigidity, this kind of cage structure enforces the effects of the so-called steric hindrance.
thereby prescribed three-dimensional structure of the functional groups,
ability to form oligomer strains,
potential applicability as artificial ion channels.

The resistance of the oligomeric 3-aminoadamantane-1-carboxylic acids based on the present invention against enzymatic cleavage is due to
(a) their pre-determined structure, which works against an “induced fit” into the active centre of the enzymes. It is known to the person skilled in the art that the Induced-Fit theory explains the formation of an enzyme-substrate complex in enzyme catalysed reactions. The Induced-Fit theory is an extension of the lock-and-key principle, according to which, a substrate (key) binds to a specific binding site in the enzyme (lock) by means of a weak, noncovalent interaction in order to stabilise the transition state. According to the Induced-Fit theory, the conformation of the enzyme is altered by the bond of the substrate, thereby enabling the specific enzyme-substrate interaction altogether. According to the Induced-Fit theory, key and lock thus alter each other. This can be found, for example, in: J M Berg, J L Tymoczko, L Stryer: “Biochemie”, Spektrum-Verlag, Heidelberg, 4th ed. 1996.
(b) their lipophilicity, which has been increased considerably in comparison to α-peptides due to the voluminous aliphatic adamantane moiety;
(c) the clearly increased distance, in comparison to α-peptides between two amide units.

The 3-aminoadamantane-1-carboxylic acid derivatives based on the present invention are suitable as both protein-based ion channels and ionophore-based ion channels as they have the following key characteristics:
(a) amphiphilicity;
(b) aggregation to tube-like structures;
(c) lipophilic on the exterior, wherein exterior refers to that side of the peptide strain seen in the direction of the helical axis of the helix with the greater distance to the helical axis, or, in the case of cyclic peptides, that side with the greater circumference;
(d) thereby, favoured integration into the lipid bilayer of cell membranes;
(e) stacking of the peptides based on the present invention if they are cyclic, (cf. general structural formula, p. 15), with the stack formed representing the artificial ion channel;
(f) one or more strands of an oligopeptide are formed by a helix or double helix which, after inclusion into the cell membrane, forms the ion channel if the peptides based on the present invention are linear and contain 4 to 40, preferentially 16-18, units n of the adamantane derivative shown on p. 15.
(g) the helical or ionophoric structure as well as the amphiphilic properties of the oligopeptides based on the present invention, can be adjusted individually by variation of R1 and/or R2.

The monomeric or oligomeric 5,7-substituted 3-aminoadamantane-1-carboxylic acid derivatives produced by the method based on the present invention can furthermore be utilised as drugs for patients for the therapy, diagnostics, and prophylaxis of diseases, during which viral infections occur. The antiviral activity of unsubstituted and 3,5-substituted aminoadamantane derivatives is known; the present invention provides a broad spectrum of further aminoadamantane derivatives for the treatment of viral infections in humans and animals. Furthermore, it is known that some viruses—for instance, the hepatitis C virus (HCV) and the BVD virus (bovine viral diarrhea virus)—themselves form ion channels and thereby impair functions of their host cells. The monomeric or oligomeric 5,7-substituted 3-aminoadamantane-1-carboxylic acid derivatives based on the present invention can act here as both antiviral and artificial ion channels and thereby eliminate the functionality of viruses.

Furthermore, the monomeric or oligomeric 5,7-substituted 3-aminoadamantane-1-carboxylic acid derivatives produced by the method according to the present invention can be used as pharmaceuticals for patients for the therapy, diagnosis and prophylaxis of diseases, wherein infections by protozoans of the genus Plasmodium occur. Plasmodia represent the causative organisms of malaria. A person skilled in the art knows of four species of plasmodia causing malaria in humans: Plasmodium falciparum, Plasmodium ovale, Plasmodium vivax, and Plasmodium malariae. Further plasmodia species known to the persons skilled in the art are, for example, Plasmodium yoeli nigeriensis, Plasmodium vinckei petteri, Plasmodium berghei yoelii, Plasmodium gallinaceum, Plasmodium gallinaceum II and Plasmodium relictum. The antiprotozoic effect of adamantane derivatives is known; the invention at hand provides a broad spectrum of further adamantane derivatives for the production of pharmaceuticals for the diagnosis, prophylaxis and therapy of plasmodia infections in humans and animals, especially for the production of pharmaceuticals against malaria infections in humans.

The monomeric or oligomeric 5,7-substituted 3-aminoadamantane-1-carboxylic acids based on the present invention are y-amino acids in a classical sense and therefore also suited as modules in peptidic catalysts. This suitability is based on the following properties:
(a) Strong hindrance of rotation around the peptidic dihedron angles φ and ψ due to an increased steric hindrance, in comparison to peptides consisting of proteinogenic amino acids, and due to the arrangement of the functional groups predetermined by the conformationally fixed adamantane skeleton: Both factors increase the stability of the secondary structure of peptidic catalysts containing the 3-aminoadamantane-1-carboxylic acid derivatives based on the present invention.
(b) The adamantane skeleton takes up considerably more room than C_{α} in proteinogenic amino acids.

The simplest y-amino acid, y-amino butyric acid (GABA), is the most important inhibiting neurotransmitter in the central nervous system. The close structural analogy is apparent.

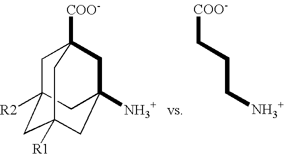

The lipophilicity, increased in comparison to GABA, of the 5,7-substituted 3-aminoadamantane-1-carboxylic acids based on the present invention facilitates overcoming the blood brain barrier; the predetermined arrangement of the functional groups to each other and the variation range in R1and R2 allow for the specific interaction/blocking of different receptor and pump systems. Furthermore, the 5,7-substituted 3-aminoadamantane-1-carboxylic acids based on the present invention feature increased protease stability; they are therefore more stable in vivo than other γ-amino acids which do not feature this conformationally rigid adamantane skeleton. Therefore, if the 5,7-substituted 3-aminoadamantane-1-carboxylic acids based on the present invention have a GABAergic effect, they can be utilised for patients for the therapy, diagnostics and prophylaxis of diseases involving a dysfunction of the GABA system, such as Morbus Parkinson, Chorea Huntington, Morbus Alzheimer, autism, Tourette syndrome, hypertension, sleep disorders, ADHD (attention deficit hyperactivity disorder), psychoses, panic and anxiety disorders, posttraumatic stress syndrome, bipolar affective disorders such as manic depressive disorders, schizophrenia.

The expression “patient” refers to humans and vertebrates alike. Thus, the pharmaceuticals can be applied in both human and veterinary medicine. Pharmaceutically acceptable compositions of compounds according to the claims are available as monomers up to oligomers or as salts, esters, amides or “prodrugs” thereof. This is provided that reliable medical evaluations do not indicate exceeding toxicity, irritations or allergic reactions when said compositions are applied. “Prodrug” is used here to refer to an active ingredient which is administered as a parent drug and which is transformed enzymatically into an active ingredient in the organism. The therapeutically active compounds of the present invention may be applied to patients either in oral, rectal, parenteral, intravenous, intramuscular, subcutaneous, intracisternal, intravaginal, intraperitoneal, intravascular, intrathecal, intravesical, topic, local (powder, ointment or drops), or spray form (aerosol). Regular dosing or application intravenously, subcutaneously, intraperitoneally or intrathecally may be carried out by means of a pump or dosing unit. Pharmaceutical forms for local application of the compounds included in the current invention comprise of ointments, powders, suppositories, sprays, and means for inhalations. Hereby, the active compound is mixed under sterile conditions according to the pharmaceutical's requirements, with a physiologically active carrier, as well as possible preservatives, buffers, diluents, and blowing agents.

### EMBODIMENTS

### Embodiment 1:

1-acetamido-3,5-dimethyladamantane via bromine-free, direct acetamidation of 1,3-dimethyladamantane

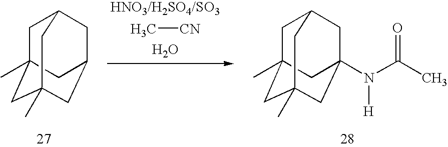

In a 100 mL round bottom flask, 1.643 g (10 mmol) of 1,3-dimethyladamantane, 12.5 mL of conc. HNO₃ (64-65%) and 15 mL of conc. H₂SO₄ (95- 98%) are mixed at 0° C. After stirring for 10 minutes at 0° C., 12.5 mL of oleum (30% SO₃) are added. Subsequently, the mixture is stirred for 1 h at 0° C. and for 3 h at room temperature. After it has cooled to 0° C. again, within 10 min 10 mL of acetonitrile are added. Subsequently, the mixture is stirred for 10 min at 0° C. and for 3 h at room temperature. Subsequently, the reaction mixture is poured onto 600 g of ice. Subsequently, extraction is carried out with diethylether (4×50 mL), the combined organic phases are dried with Na₂SO₄ and the ether is removed in the rotary evaporator. The crude product is taken up with dichloromethane and filtered with a glass frit over 20 g of basic Al_{2O}₃. Rotary evaporation of dichloromethane at 15 mbar and 60° C. yields 1.330 g (60%) of the 1-acetamido-3,5-dimethyladamantane as a colourless solid.

### ¹H—NMR (400 MHz, CDCl₃, TMS): δ=0.84, s, 6H; 1.15, m, 2H; 1.33, m, 4H; 1.63, 4H; 1.82, m, 2H; 1.90, s, 3H; 2.13, m, 1H; 5.23, bs, 1H.

### ¹³C—NMR (100 MHz, CDCl₃, TMS): δ=24.65(+); 30.02(+); 30.12(+); 32.33(0); 40.16(−); 42.67(−); 47.61(−); 50.61(−); 53.43(0); 169.23(0).

MS (m/z): 221; 164; 150; 122; 107; 91.

### Embodiment 2:

1-amino-3,5-dimethyladamantane hydrochloride

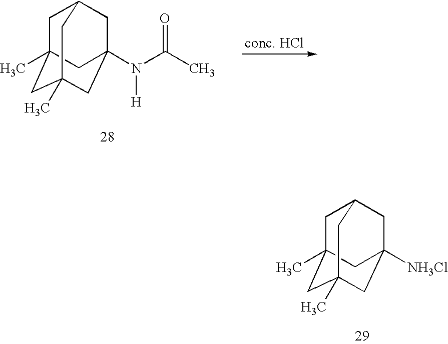

In a 25 mL round bottom flask, 1 mmol of 1-acetamido-3,5-dimethyladamantane and 15 mL of conc. HCl (36-38%) are refluxed for 20 h. After cooling them to room temperature, the excess hydrochloric acid is removed until dryness by a vacuum distillation (15 mbar, 90° C.).

Recrystallisation of the colourless remainder from water yields 140 mg (65%) of the 1-amino-3,5-dimethyladamantane-hydrochloride in the form of a colourless solid.
¹H—NMR (400 MHz, d₆-DMSO, TMS): δ=0.84, s, 6H; 1.14, m, 2H; 1.29, m, 4H; 1.48, m, 4H; 1.67, m, 2H; 2.14, m, 1H; 7.46, bs, 3H.
¹³C—NMR (100 MHz, d₆-DMSO, TMS): δ=29.03(+); 29.53(+); 33.43(0); 38.39(−); 41.46(−); 45.79(−); 49.46(−); 52.29(0).

### Embodiment 3

1-bromo-3-iodo-5,7-dimethyladamantane

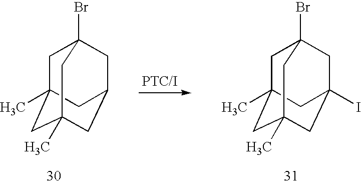

In a 250 mL round bottom flask, 2.4319 g (10 mmol) of 1-bromo-3,5-dimethyladamantane, 15.76 g (40 mmol) of iodoform, 484 mg (1.5 mmol, 15 mol-%) of tetra-n-butyl ammonium bromide, 10 g of solid NaOH and 80 mL of fluorobenzene are mixed. This flask is provided with a reflux condenser and suspended in an ultrasonic bath of Bender&Hobein Laboson 200 (35 kHz). At 80° C. (temperature of the silicon oil in the ultrasonic bath), the reaction is carried out. After 3 d, 4 d, 5 d, 6 d and 7 d of reaction time, respectively, sucking off over a glass frit (Schott, G4, pore width 10-16 μm) is carried out, then washed three times with 30 mL of diethylether each, the solvents are distilled until dryness in the rotary evaporator at reduced pressure (p=15 mbar), and after GC-MS analysis, the reaction is started again with the same quantities of reagents and solvents. After 8 d, only traces of the educt 1-bromo-3,5-dimethyladamantane remain via GC/MS-analysis so that the reaction is ended and the crude product obtained is subjected to a separation via column chromatography (SiO₂, J. T. Baker, 0.063-0.200 mm; 3.0×30 cm, n-pentane, R_{f}=0.31). Rotary evaporation of the eluent at room temperature and 15 mbar yield 2.1913 g (59%) of the bromoiodoadamantane in the form of a colourless solid.

### ¹H—NMR (400 MHz, CDCl₃, TMS): δ=0.89, s, 6H; 1.23-1.38, m, 2H; 2.03, bs, 4H; 2.14-2.60, m, 4H; 2.90, bs, 2H.

### ¹³C—NMR (100 MHz, CDCl₃, TMS): δ=28.85(+); 39.12(0); 43.07(0); 47.94(−); 53.09(−); 55.98(−); 60.05(−); 61.52(0).

IR (KBr-pellet): 2950.9, 2923.9, 2863.0, 2840.6, 1452.1, 1441.6, 1350.9, 1334.0, 1315.2, 1230.1, 1167.4, 891.0, 828.1, 703.3cm⁻¹.

### MS m/z (%): 289(4)M⁺—Br; 241/243, (100)M⁺—I; 201(1); 185(16); 173(3); 161(42); 145(2); 133(3); 119(29); 91(29); 79(13); 69(7); 55(16); 42(14).

EA C₁₂H₁₈Brl (369.1): calc. C 39.05 H 4.91 found C 39.14 H 4.72.

### Embodiment 4:

N1-(3-bromo-5,7-dimethyl-1-adamantyl) acetamide

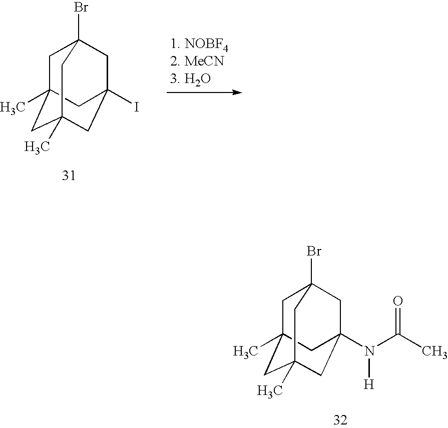

In a pre-heated 250 mL two neck flask with an inside low temperature thermometer and a dropping funnel, 467.2 mg NOBF₄ (4 mmol) are dissolved in 10 mL of absolute acetonitrile under dry argon and cooled to an inside temperature of

−50° C. with an acetone-dry ice bath. At an inside temperature of −40° C. up to −50° C., a solution of 1.4764 g (4 mmol) of 1-bromo-3-iodo-5,7-dimethyladamantane in 70 mL of water-free acetonitrile is added dropwise within 30 minutes. While stirring, the reaction mixture is warmed to −10° C. within 3 hours. At −30° C., the reaction begins, as can be seen in a brown colouration and gas formation. At −10° C., the gas formation is complete.
20 mL of water and 30 mL of diethylether are added; then, the phases are separated and the aqueous phase is extracted with diethylether (3 times 25 mL). The combined etheric phases are washed with NaHSO₃ solution, water, and saturated saline solution (2 times 20 mL, resp.) and dried with Na₂SO₄. After filtering the desiccant and removing the solvents at the rotary evaporator in vacuo (15 mbar), purification by column chromatography takes place (SiO₂, J. T. Baker, 0.063-0.200 mm; 2.0×50 cm, diethylether, R_{f}=0.28). The rotary evaporation of the eluent at 15 mbar and 40° C. yields 984.7 mg (82%) of the bromoacetamide in the form of a colourless solid.

### ¹H—NMR (400 MHz, CDCl₃, TMS): δ=0.92, s, 6H; 1.13-1.24, m, 2H; 1.57-1.77, m, 4H; 1.87-2.03, m, 4H and 1.91, s, 3H, 2.45, bs, 2H, 5.24, bs, 1 H.

### ¹³C—NMR (100 MHz, CDCl₃, TMS): δ=24.496(+), 29.00(+), 35.77(0), 45.96 (−),48.83(−), 50.78(−), 53.96(−), 55.27(0), 62.68(0), 169.35(0).

IR (KBr pellet): 3294.7, 3080.6, 2947.1, 2927.8, 2901.3, 2864.7,1676.3, 1653.9, 1557.5, 1442.5, 1369.7, 1322.9, 1302.6, 1189.5, 874.8, 741.7, 605.8cm⁻¹.

### MS m/z (%): 301/299(3) M+; 259(2); 220(100); 200(3); 178(20); 164(27); 145(2); 121(26); 105(10); 91(7); 79(5); 58(16); 41(40). M_{abs.}299.088475, M_{found}299.0906.

EA C₁₄H₂₂BrNO (300.23): calc C 56.00 H 7.38 N 4.66, _{found} C 55.85 H 7.35 N 4.59.

### Embodiment 5:

3,5-dimethyl-7-methylcarboxamido-1-adamantane carboxylic acid

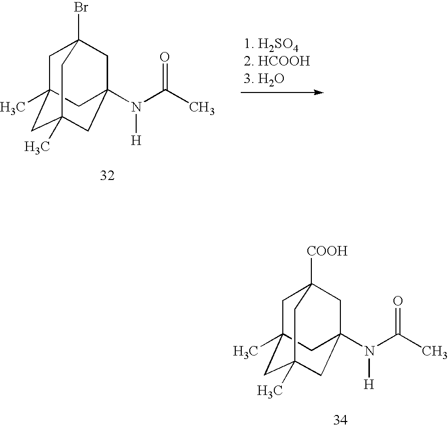

Although the substance is known from scientific publications to date it has merely been characterised by melting point and elementary analysis; here, it is produced in a new manner based on the present invention. In a 250 mL round bottom flask fitted with a dropping funnel, 600 mg (1.998 mmol) of acetamide are dissolved in 80 mL of conc. H₂SO₄ (95-98%). The solution takes on a reddish-brown colouring. Now, 20 mL of formic acid (98-100%) are added dropwise at room temperature over a course of 4 h. After this is finished, more stirring takes place until the end of the gas formation (approx. 30 min.). Then, the reaction mixture is poured onto 600 g of ice and left standing for 2 h. A colourless precipitate forms which is sucked off. It is dissolved with a 10% aqueous sodium hydroxide solution, filtered and precipitated again by acidification with conc. HCl to pH=3. Renewed sucking off and recrystallisation from glacial acetic acid/water/acetone (5:5:4) yields 329 mg (62.1%) of the acetamidocarboxylic acid in the form of colourless, leafy crystals.

¹H—NMR (400 MHz, d₆-DMSO, TMS): δ=0.85, s, 6H; 1.02-1.11, m, 2H; 1.31-1.42, m, 4H; 1.46-1.58, m, 4H; 1.73, s, 3H; 1.85, bs, 2H, 7.41, s, 1H; 12.1, bs, 1H.
¹³C—NMR (100 MHz, d₆-DMSO, TMS): δ=23.65(+), 29.53(+), 31.93(0), 40.87(−), 42.77(0), 43.94(−), 46.21 (−), 49.32(−), 52.37(0), 168.73(0), 177.51(0).
IR (KBr pellet): 3339.6, 3088.6, 2942.2, 2897.5, 2868.5, 2848.3, 2478.1, 1971.3, 1687.3, 1609.5, 1552.9, 1455.3, 1372.3, 1313.3, 1270.7, 1261.1, 1225.0, 1197.2, 846.1, 177.3cm⁻¹.
MS m/z(%): 265(100) M⁺, 250(1), 237(2), 220(52), 194(15), 178(9), 164(61), 150(17), 137(3), 122(20), 107(15), 91(13), 77(7), 55(13), 41(68); M_{abs.}265.16779, M_{found}265.1708.
EA C₁₅H₂₃NO₃ (265.35): calc C 67.89 H 8.73 N 5.27, found C 67.35 H 8.82 N 4.93.

### Embodiment 6:

3-carboxy-5,7-dimethyl-1-adamantane ammonium chloride

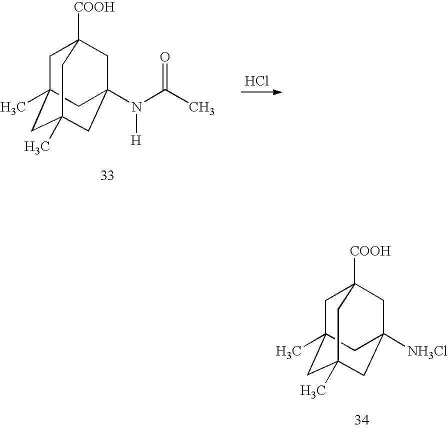

This substance is produced according to a modified regulation by Stepanov et al. (F. N. Stepanov, Y. I. Srebrodolskii, J. Org. Chem. USSR 1966, 2, 1612-1615). For this purpose, 6.5 g (24.5 mmol) of the acetamide are refluxed 3 d in a 250 mL round bottom flask with 155 conc. HCl. After cooling, the reaction mixture is evaporated to dryness at the rotary evaporator at 100-400 mbar and 90° C.

The remainder is digested with 40 mL of ice-cold acetone, sucked off, and washed with 20 mL of ice-cold acetone. 5.1031 g (81%) of the hydrochloride are obtained in the form of a colourless solid.
¹H—NMR (400 MHz, d₆-DMSO, TMS): δ=0.89ppm, s, 6H; 1.05-1.20, m, 2H; 1.31-1.53, m, 8H; 1.77, bs, 2H; 7.32, t, J=51.4 Hz (¹H-¹⁵N-quadrupole coupling) and 8.34, bs, overall 3H; 12.3, bs, 1H.
¹³C—NMR (100 MHz, d₆-DMSO, TMS): δ=28.92(+), 32.01(0), 40.12(−), 42.61(0), 43.18(−), , 44.91(−), 48.40(−), 52.47(0), 176.62(0).

### Embodiment 7:

3-(9-fluorenylmethoxycarbonylamido)-tricyclo[3.3.1.1^{3,7}]decan-1-carboxylic acid

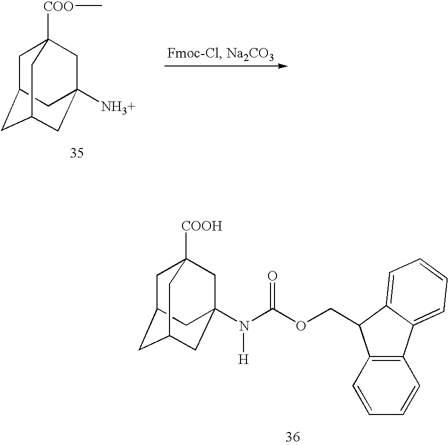

In a 100 mL round bottom flask, 1.5621 g (8 mmol) of zwitter ion are mixed with 24 mL of 10% aqueous Na₂CO₃ solution and 20 mL of acetone. The mixture is cooled to 0° C. in an ice bath, followed by the dropwise adding of 2.0696 g (8 mmol) of Fmoc-Cl in 20 mL of acetone within 30 minutes. Subsequently, stirring occurs for 1 h at room temperature and then for 1 h at 50° C.

Approx. 40 mL of the mixture are then evaporated in the rotary evaporator at 50 mbar and 50° C. and the remaining reaction mixture is poured into 250 mL of cold water. It is acidified to pH=3 with 2 N HCl and extracted with acetic acid ethyl ester (3 times 25 mL). After drying with anhydrous Na₂SO₄, filtering and evaporating to dryness by rotary evaporation at 15 mbar and 60° C., 2.774 g of crude product remain, which are recrystallised from approx. 40 mL of nitromethane. After standing 10 days at 4° C. up to 8° C. for the completion of crystallisation, 2.004 g (60%) of the Fmoc-protected amino acid are obtained in the form of faintly yellowish crystals.
¹H—NMR (400 MHz, d₆-DMSO, TMS): δ=1.4-2.2, m, 14H; 3.3, bs, 1 H; 4.2, t, J=6Hz, 1H; 4.35, m, 2H; 4.70, bs, 1H; 7.32, dt, J=7.5/1 Hz, 2H; 7.38, m, 2H; 7.58, d, J=7.5Hz, 2H; 7.75, d, J=7.5Hz, 2H; 12.1, bs, 1H.
¹³C—NMR (100 MHz, d₆-DMSO, TMS): δ=28.54(+); 34.88 (−); 37.59(−); 40.22(−); 41.53(0); 42.37(−); 46.81(+); 50.13(0); 64.76(−); 119.93(+); 125.06(+); 126.92(+); 127.46(+); 140.70(0); 143.98(0); 154.27(0,weak); 177.45(0)
MS m/z(%): 417(2), M⁺, 368(2), 230(2), 208(3), 192(4), 178(100), 165(10), 150(2), 138(5), 128(4), 93(8), 77(4), 65(5), 57(4), 40(30); M_{abs}=417.19401,

### M_{fould}=417.1900

IR (KBr pellet): 3318.4, 3067.7, 2913.3, 2855.9, 1719.2, 1677.0, 1556.2, 1449.7, 1264.1, 1090.6, 732.8, 725.5 cm⁻¹
EA C₂₆H₂₇NO₄(417.5): calc: C 74.52, H 6.52, N 3.39; found: C 74.63, H 6.57, N 3.41.

### Embodiment 8:

1,1-dimethylethyl-3-aminotricyclo[3.3.1.13,7]-decane-1-carboxylate

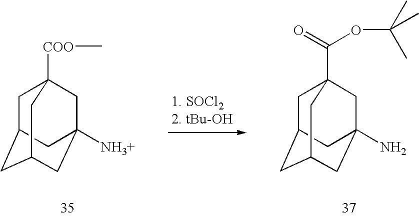

In a 100 mL round bottom flask, 1.562 g (8 mmol) of zwitter ion are refluxed with 50 mL of thionyl chloride for 100 min (76° C.). After the evaporation of the excess thionyl chloride in the rotary evaporator at 15 mbar and 60° C. to dryness by rotary evaporation, the remainder is refluxed with 50 mL of tert.-butanol for a further 100 min (82° C.-83° C.). The remainder after the evaporation of the excess alcohol to dryness by rotary evaporation at 15 mbar and 80° C. is taken up in 40 mL of saturated aqueous Na₂CO₃ solution and extracted with diethylether (4 times 50 mL). The combined etheric extracts are dried with Na₂SO₄, the desiccant is filtered and the filtrate is evaporated to dryness in the rotary evaporator at 15 mbar and 50° C. 1.9235 g (96%) of the ester are obtained as an NMR pure, slightly yellowish oil. Subsequently, the crude product is subjected to separation by column chromatography (SiO₂, J. T. Baker, 0.063-0.200 mm; dichloromethane/triethylamine 95:5, 3.0×30 cm, R_{f}=0.29).

### ¹H—NMR (400 MHz, CDCl₃, TMS): δ=1.29, m, 2H; 1.42, s, 9H; 1.50-1.60, m, 6H; 1.65, s, 2H; 1.68-1.79, m, 4H; 2.10-2.20, m, 2H.

### ¹³C—NMR (100 MHz, CDCl₃, TMS): δ=27.84(+); 29.36(+); 35.11(−); 37.62(−); 43.28(0); 45.02(−); 47.35(−); 47.52(0); 79.42(0); 176.07(0).

MS m/z(%): 251(37) M⁺; 208(1); 194(19); 178(4); 150(100); 138(37); 127(9); 108(9); 94(68); 77(7); 57(53); 41(22). M_{abs.}=251.18853, M_{fould}=251.1905.
IR (Film): 3358.4, 2976.8, 2903.9, 2852.3, 1718.3, 1591.4, 1454.4, 1367.2, 1267.4, 1174.1, 1144.3, 853.3 cm⁻¹.
EA C₁₅H₂₅NO₂ (251.36): calc C 71.76 H 10.02 N 5.57 found C 71.29 H 10.26 N 5.77.

### Embodiment 9:

1,1-dimethylethyl-3-[3-(9-fluorenylmethoxy-carbonylamino)-tricyclo [3.3.1.13,7]dec-1-ylcarboxamido]-tricyclo[3.3.1.13,7]decan-1-carboxylate

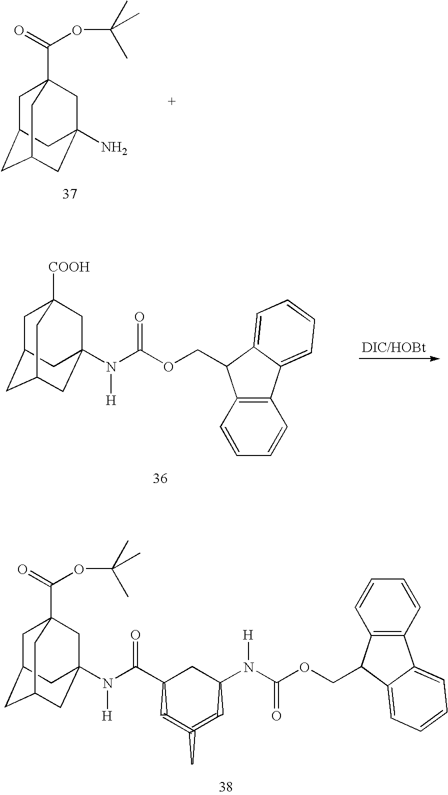

In a pre-heated 250 mL round bottom flask fitted with a dropping funnel, a total of 711 mg (5.6 mmol) of diisopropylcarbodiimide is added within 5 minutes in five portions of approx 140 mg each, under dry argon, to a solution of 2.3502 g (5.63 mmol) of the Fmoc-protected amino acid as well as 726.2 mg (5.63 mmol) of HOBt in 60 mL of anhydrous THF at 0° C. while cooling in an ice bath. Further stirring occurs for 30 min. at 0° C.; subsequently, within approx. 45 minutes, a solution of 1.4161g (5.63 mmol) of the tBu-ester in 30 mL of anhydrous THF is added dropwise. The solution is heated to room temperature within 4 h and stirred for 7 d at room temperature. Then, the THF is removed until dryness in the rotary evaporator at 15 mbar and 70° C., thereafter, take-up with 60 mL of diethylether and 25-fold extraction, each with 30 mL of water, is carried out. Subsequently, the diethylether is removed until dryness in the rotary evaporator at 15 mbar and 60° C. The crude product thus obtained is subjected to purification by column chromatography (SiO₂, J. T. Baker, 0.063-200 mm; 2.5×50 cm, chloroform/methanol 98:2, R_{f}=0.80). Evaporation of the eluent at 15 mbar and 60° C., and drying in the desiccator over P₂O₅ at 15 mbar yields 3.006 g (82%) of the dipeptide in the form of a faintly yellowish foam.

¹H—NMR (400 MHz, CDCl₃, TMS): δ=1.42, s, 9H; 1.50-1.70, m, 4H; 1.70-1.82, m, 8H; 1.82-2.05, m, 12H; 2.12-2.30, m, 4H; 4.18, t, J=6.5Hz, 1H; 4.254.40, m, 2H; 4.68-4.75, m, 2H; 7.31, dt, J=7.5/1 Hz, 2H; 7.38, t, J=7.1 Hz, 2H; 7.57, d, J=7.3Hz, 2H; 7.75, d, J=7.3Hz, 2H.
¹³C—NMR (100 MHz, CDCl₃, TMS): δ=27.95(+); 29.13(+); 29.17(+); 35.28(−); 37.91(−); 38.18(−); 40.52(−); 40.65(−); 40.76(−); 42.53(−); 42.90(0); 43.02(0); 43.11(−); 47.26(+); 51.22(0); 51.68(0); 65.90(−); 79.83(0); 119.90(+); 124.93(+); 126.98(+); 127.59(+); 141.28(0); 143.90(0); 154.30(0); 175.70(0); 175.80(0).
MS m/z(%): 321(4); 279(5); 251(27); 236(11); 194(12); 178(20); 167(10; 150(100); 138(26); 119(4); 108(7); 94(47); 70(11); 57(37); 41(17).
IR (KBr pellet): 3357.5, 3065.7, 2909.2, 2855.7, 1718.3, 1649.4, 1512.9, 1450.9, 1366.7, 1252.2, 1167.4, 1081.4, 740.2cm⁻¹.
EA C₄₁H₅₀N₂O5 (650.85): calc. C 75.66 H 7.74 N 4.30 found C 75.18 H 7.80 N 4.41.

### Embodiment 10:

Analytical enantiomer separation of (+)-3-acetamido-5-methyladamantan-1-carboxylic acid tert.-butyl ester

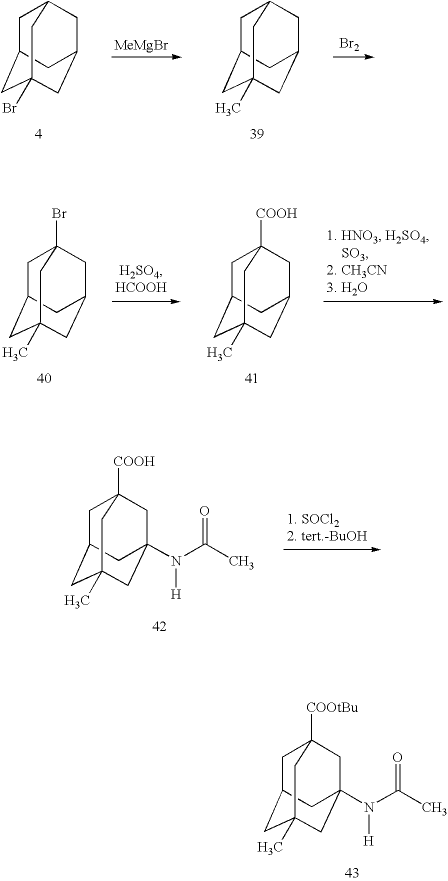

Here, first the racemate of the aforementioned compound, based on 1-bromoadamantane, shall be described.

a.) Synthesis of 1-methyladamantane 39:

The production is carried out according to a procedure known from literature (G. Molle, J. E. Dubois, P. Bauer, Can. J. Chem. 1987, 65, 2428-2433). For its purpose, 400mmol of 1-bromoadamantane (=86.056 g) and 50 mL of di-n-butylether dried over sodium wire are provided in a pre-heated 2,000 mL three neck round bottom flask fitted with a dropping funnel and a reflux condenser under dry argon and heated to an oil bath temperature of 105° C. Then, within 90 minutes, 800 mL of a 1 N solution of methylmagnesiumbromide in di-n-butylether are added dropwise. After adding it, stirring is continued for an additional 3 h in an oil bath at a temperature of 105° C. After cooling to room temperature, the excess of the Grignard reagent is hydrolysed by adding 1 N hydrochloric acid until an acidic reaction occurs. The phases are separated, the organic phase is washed twice, with 50 mL each of saturated NHCO₃ solution, twice with 50 mL each of water, and twice with 50 mL each of saturated NaCl solution, and dried with anhydrous Na₂SO₄. After filtering the desiccant, the solvent is distilled via a vacuum distillation over a 20 cm Vigreux column. The remainder is purified by column chromatography (SiO₂, J. T. Baker, 0.063-0.200 mm; 5.0×100 cm, n-pentan, R_{f}=0.95). Rotary evaporation of the eluent at 0° C. and 15 mbar yields 47,937 g (80%) of the 1-methyladamantane in the form of a colourless solid. ¹H—NMR (400 MHz, CDCl₃, TMS): δ=0.75, s, 3H; 1.43, d, J=2.4Hz, 6H; 1.55-1.70, m, 6H; 1.86-1.93, m, 3H; this data is congruent with literature.

¹³C—NMR (100 MHz, CDCl₃, TMS): Not reported in the original literature. δ=28.90(+); 29.81(0); 31.44(+); 36.94(−); 44.66(−).

b.) Synthesis of 1-bromo-3-methyladamantane 40:

This regulation was adopted from literature (J. Applequist, P. Rivers, D. E. Applequist, J. Am. Chem. Soc. 1969, 91, 5705-5711) and modified. 53.3 g (333 mmol) of freshly distilled bromine are slowly added dropwise, while stirring and cooling in an ice bath, to 5 g (33.3 mmol) of 1-methyladamantane in a 100 mL round bottom flask.

HBr gas is formed. After completing the adding of bromine, the mixture is stirred, first for 30 min. at room temperature, then for 90 min. under reflux. Subsequently, approximately half of the bromine is distilled and the remaining mixture is added dropwise, slowly, while stirring, to an oversaturated solution of NaHSO₃ in ice water. The overall resulting solution is extracted with ether four times, the combined ether phases are washed twice with 50 mL of saturated NaHSO₃ solution, then twice with 50 mL of water and twice with 50 mL of saturated saline solution. After drying with Na₂SO₄, filtering occurs and the ether is distilled at room temperature and 15 mbar. This yields 7.631 g (=100%) of the bromomethyladamantane in the form of a faintly yellowish liquid which rigidifies after 1-4 hours of standing at room temperature (c. 21° C.) (Fp=22-24° C.).
¹H—NMR (400 MHz, CDCl₃, TMS): δ=0.85, s, 3H; 1.46, d, 4H; 1.62, m, 2H; 2.08ppm, s, 2H; 2.12, m, 2H; 2.27, m, 4H.

### ¹³C—NMR (100 MHz, CDCl₃, TMS): δ=30.18(+); 32.72(+); 34.77(−); 35.25(0); 42.55(−); 48.49(−); 55.98(−); 66.57(0).

c.) Synthesis of 3-methyladamantane-1-carboxylic acid 41:

A regulation from literature (J. Applequist, P. Rivers, D. E. Applequist, J. Am. Chem. Soc. 1969, 91, 5705-5711) is modified:

5.5 g (24 mmol) of 1-bromo-3-methyladamantane are dissolved in a 250 mL round bottom flask in 100 mL of conc. H₂SO₄ and 15 mL of oleum (30% SO₃). Hereby, the conc. sulphuric acid is added first; then, within 1 up to 5 minutes, the oleum is added while stirring. 40 mL of conc. formic acid are added dropwise within 3 hours at room temperature while stirring; Hereby, CO is released under heavy foaming. After completely adding of formic acid, stirring at room temperature continues until the end of the gas formation (approx. 1 h). Subsequently, the reaction mixture is poured onto approx. 800 g of ice and left standing for 1 h. A colourless precipitate forms which is isolated by sucking off. The crude product is dissolved in 10% NaOH and filtered; the filtrate is acidified strongly with conc. HCl up to pH 1-3 and left standing for 1 h; then, the pure product is sucked off. Washing with cold water and drying in the desiccator over P₂O₅ for 24-72 h i. vac. yields 3.9631 g (85%) of the 3-methyl-1-adamantanecarboxylic acid in the form of a colourless solid.
¹H—NMR (400 MHz, d₆-DMSO, TMS): δ=0.8, s, 3H; 1.4, m, 4H; 1.5, s, 2H; 1.57, m, 2H; 1.69, m, 4H; 2.0, m, 2H.
¹³C—NMR (d₆-DMSO, TMS): δ=27.97(+); 29.60(0); 30.62(+); 35.22(−); 37.80(−); 40.44 (0); 43.00(−); 45.29(−); 178.21(0).

d.) Synthesis of 3-methyl-5-(methylcarboxamido)-1-adamantane carboxylic acid 42:

The product is known from literature; however, in literature (F. N. Stepanov, Y. T. Srebrodol'skii, Zh. Org. Khim. 1966, 9, 1612-1615) it is produced from 1-acetamido-3-bromo-5-methyladamantane via a Koch-Haaf reaction.

While cooling in an ice bath, 15 mL of conc. HNO₃ are added to 3.796 g (19.54 mmol) of 3-methyladamantane-1-carboxylic acid in a 250 mL round bottom flask. Subsequently, 20 mL of conc. H₂SO₄ are added within 0.5 to 5 minutes. A red colouration of the reaction mixture occurs. Stirring is continued for 10 minutes at 0° C., after which, within 0.5 to 5 minutes, 16 mL of oleum (20% SO₃) are added. Stirring continues for 1 h at 0° C., then for 3 h at room temperature. After renewed cooling to 0° C., 15 mL of acetonitrile are added dropwise within 1 up to 20 minutes. Stirring for 10 min. at 0° C. and 3 h at room temperature completes the reaction.
After stirring, the reaction mixture is poured onto approx. 500 g of ice. After standing 30 minutes, a colourless precipitate forms which is sucked off. The precipitate is dried in the drying pistol at 90° C. i. vac (15 mbar) within 10 to 15 hours. 4.6652 g (95%) of the acetamide are yielded, which is recrystallised from glacial acetic acid/water/acetone (5 : 5 : 3). 3.8918 g (=85%) of the pure product are yielded in the form of a colourless solid.
¹H—NMR (400 MHz, d₆-DMSO, TMS):δ=0.83, s, 3H; 1.32, m, 2H; 1.43, s, 2H; 1.60, m, 4H; 1.74, s, 3H; 1.77, m, 2H; 1.92, s, 2H; 2.11, m, 1H; 7.4, s, 1H.

### ¹³C—NMR(d₆-DMSO, TMS): δ=23.66 (+); 28.90(+); 29.90(+); 31.35(0); 37.00(−); 39.34(−); 40.13(0); 41.50(−); 42.06(−); 44.53(−); 46.90(−); 51.59(0); 168.70(0); 177.54(0).

e.) Synthesis of 3-acetamido-5-methyladamantane-1-carboxylic acid tert.-butylester 43:

In a pre-heated 100 mL two neck round bottom flask fitted with a septum and a reflux condenser, 1.4276 g of thionylchloride (=12 mmol) are added, under dry argon, to 2.5115 g of 3-acetamido-5-methyladamantane-1-carboxylic acid (=10 mmol) in 50 mL of water-free tetrahydrofurane passing through the septum with a medical syringe at room temperature. It is refluxed for 10-15 hours. After cooling to room temperature, 15 mL of tert.-butanol are added, and refluxing occurs for an additional 3 h. After renewed cooling, the mixture is washed with water until it is acid-free (pH 6-8), dried with anhydrous Na₂SO₄, the desiccant is filtered, and the volatile components of the reaction mixture are removed via vacuum distillation (15 mbar, 75° .C). The faintly yellowish oil thus yielded is purified by column chromatography (SiO₂, J. T. Baker, 0.063-0.200 mm; 3.0×30 cm, tert.-butyl-methylether, R_{f}=0.31)

¹H—NMR (40 MHz, d₆-DMSO, TMS):δ=0.94, m, 3H; 1.44, s, 9H; 1.4-2.3, m, 13H; 2.0, s, 3H; 6.3, bs, 1H.

### ¹³C—NMR(d₆-DMSO, TMS): δ=27.65(+); 27.98(+); 29.46 (+); 29.88 (+); 32.14(0); 38.52 (−); 39.54 (−); 41.38(−=; 41.62(−); 41.80(0); 44.01(−); 46.11(−); 53.91(0); 80.15(0); 162.50(0); 175.40(0).

MS (m/z): 307, 249, 206, 193, 175, 147,107, 57.

The tert.-butylester thus produced is separated into its enantiomers on an HPLC column, by Co. Macherey-Nagel (Nucleodex® β-PM, 4 mm×150 mm, grit size 5 μm, eluent: methanol/water (70: 30).

### Embodiment 11:

Uptake of γ-aminoadamantane carboxylic acid derivatives 44-51 by the mGAT1 transporter in Xenopus laevis oocytes

### Preparation of Oocytes

Female African claw frogs (Xenopus laevis) are anaesthetised with Tricaine (MS222, Sandoz, Basel/Switzerland, 1 g L⁻¹). Parts of the ovary are removed and treated with collagenase in order to remove follicle cells. For the experiments, full-grown oocytes are selected. For the expression, cRNA of the GABA transporter GAT1 from mouse brain is injected (approx. 50 ng per oocyte). These oocytes, as well as untreated control oocytes into which no cRNA is injected, are incubated for 3 days at 19° C. in oocyte Ringer solution ORi (composition in mM: NaCl 90, KCl 2. MgCl₂ 2, MOPS (morpholinopropane sulphonic acid)/Tris 5 (adjusted to pH 7.4), Gentamycin (70 mg L⁻¹).

### Uptake Measurement of the GABA Analogues.

The following substances are utilised:

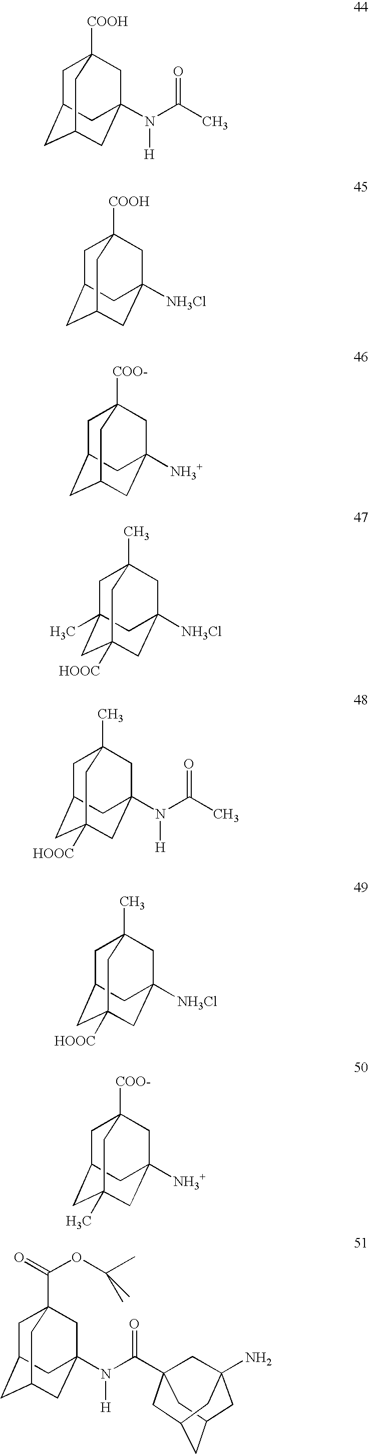

In each case, the uptake of the substances is measured by incubating 10 of the oocytes which have been pre-treated as described above in approx. 200 μl of ORi solution at room temperature (21° C.) for 20 min. The Ori utilised here contains 400 μM GABA; of which about 1 μM is ³H labelled, as well as 1 mM of substances 46-53. In stock solutions of these substances, they are solubilised by the addition of DMSO if necessary. After the incubation, the oocytes are washed and lysed with SDS solution (10% aqueous SDS in aqua bidest.). 3 mL of scintillation solution (Rotiszint Eco Plus) is added to each dissolved oocyte; they are then incubated for 2 h at 40° C., shaken, and radioactivity is measured with a scintillation counter.

In order to exclude oocytes with high membrane leakage, 1 mM of sucrose with 18 μM[¹⁴C]-sucrose (16 kBq per 200 μl, DuPont NEN, Bad Homburg) are added to the incubation medium. Only oocytes with intact membranes are utilised for the averaging. The criteria for the evaluation of membrane density in oocytes are known to the person skilled in the art and have been described, for example, in: U. Eckstein-Ludwig et al., Br. J. Pharmacol. 1999, 128, 92-102.
The uptake values have been normalised to the uptake of GABA (=1).

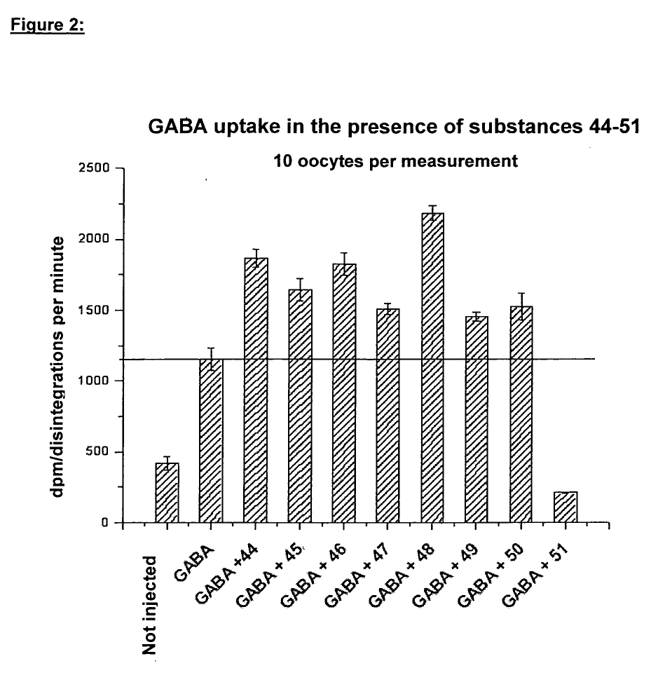

The results are illustrated in FIG. 2.

### Embodiment 12:

Inhibition of the mGAT1-mediated uptake of GABA by 1,1-dimethylethyl-3-(3-aminotricyclo[3.3.1.1 ^{3,7}]dec-1-ylcarboxamido)tricyclo[3.3.1.1^{3,7}]decan-1-carboxylate (53) in Xenopus laevis oocytes

The preparation of the oocytes, incubation with the dissolved substance 51 and further treatment correspond to the information described in Embodiment 8.

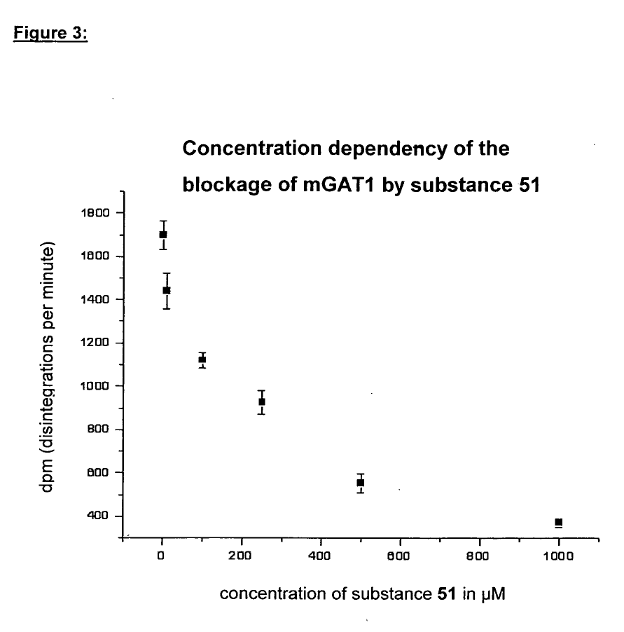

The results are illustrated in FIG. 3.

### FIGURE LEGENDS AND LIST OF REFERENCE NUMERALS

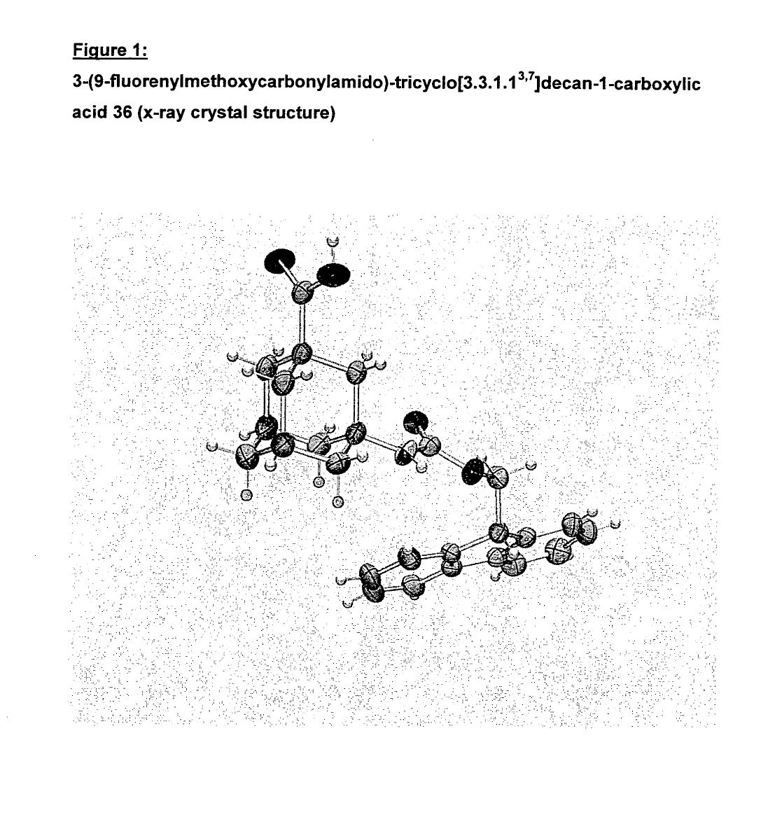

FIG. 1: x-ray structural analysis of 3-(9-fluorenylmethoxycarbonylamido)-tricyclo[3.3.1.1^{3,7}]decane-1-carboxylic acid 36
ORTEP production of 3-(9-fluorenylmethoxycarbonylamido)-tricyclo[3.3.1.13,7]decan-1-carboxylic acid, thermal ellipsoids with a 50% probability density.
FIG. 2: uptake of γ-aminoadamantane carboxylic acid derivatives by the mGat1 transporter in Xenopus laevis oocytes
Normalised uptake of GABA in the absence, and resp., presence of substances 44-51. Per measurements, 10 oocytes were utilised and the results were averaged. The concentration of the examined substances is 1000 μM in each case. Not injected: control measurement. No GABA transporter was expressed in these oocytes.
FIG. 3: Concentration dependency of the blockage of mGAT1 by substance (51)
Utilised were ORi solutions with the following concentrations in substance 51 (μM): 0, 10, 100, 250, 500, 1000. Per measurement, 10 oocytes were utilised and the results were averaged.

1. a compound having the formula 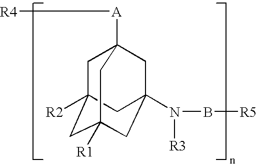
wherein
R1 and R2 are independent from each other:
H, F, Cl, Br, I;
L=-alkyl, -alkenyl, -alkinyl, -cycloalkyl, -cycloalkenyl, -heterocycloalkyl, -heterocycloalkenyl, -aryl, -heteroaryl, -alkylaryl, -alkylheteroaryl, -alkylcycloalkyl, -alkylheterocycloalkyl, -alkenylcycloalkyl, -alkenylheterocycloalkyl,
wherein -alkyl represents a group with 1 up to 10 carbon atoms, preferentially methyl, ethyl, propyl, isopropyl, 1-butyl, 2-butyl, (2-methyl-)propyl, tert.-butyl and -alkenyl and -alkinyl represent a monounsaturated or polyunsaturated group containing 2 up to 10 carbon atoms which contains, in the case of the alkenyl, at least one —C=C-bond and, in the case of the alkinyl, at least one —C≡C-bond; -alkyl, -alkenyl, and resp., -alkinyl are linear or branched, -cycloalkyl and -cycloalkenyl represent a group with 3 up to 20 carbon atoms; the heterocyclic groups represent a residue with 1 up to 20 carbon atoms, wherein up to 5 carbon atoms are substituted by heteroatoms selected from the group comprising: nitrogen, oxygen, sulphur and phosphorus, -aryl represents an aromatic residue with 5 up to 20 carbon atoms and heteroaryl represents a corresponding aromatic residue wherein up to 5 carbon atoms are substituted by heteroatoms selected from the group comprising: nitrogen, oxygen, sulphur, phosphorus,
wherein L contains optionally one up to three substituents selected from the group comprising: F; Cl; Br; I; —OH; —O—(C₁-C₁₀-alkyl); —SH; —S—(C₁-C₁₀-alkyl); —SO₃H; —CN; —COOH; —COO—(C₁-C₁₀-alkyl); —O—(C=O)——(C₁-C₁₀-alkyl); —CONH₂; —CONH(C₁-C₁₀-alkyl); —CON(C₁-C₁₀-alkyl)₂, wherein the two alkyl groups are identical or different; —NH₂, —NH(C₁-Cl₁₀-alkyl); —N(C₁-C₁₀-alkyl)₂, wherein the two alkyl groups are identical or different, and wherein alkyl groups are linear or branched,
and/or R1 and R2 are independent from each other
—OH, —O—, —OL, —SH, —S—, —SL, —SOH, —SO₂H, —SO₃H, —(S=O)-L, 1'SO₂L, —NO, —NO₂, —C≡N, —C═N-L, —N≡C, —N═C-L, —NH₂, —NHL, —NH₂L⁺, —NYZ, —NHYZ⁺, —CHO,
—COL, —COOH, —COO—, —COOL, —O(CHO), —O(C═O)L, —CONH₂, —CONHL, —CONYZ, —NHCOOH, —NLCOOH, —NLCOOL, —NHCOOL, —NH—(C═O)L, —NH—(C═N—H)—NH₂, —NH—(C═N—H)—NYZ, —NY—(C═N-Z)—NHL, —NH—(C═N—H)—NHL, —SO₂—NH-L, —SO₂—NH₂, —SO₂—NYZ, —NY—SO₂Z, —O—(CₚH₂ₚ)ₓ—O-L; —(CₚH₂ₚ)ₓ—O-L; —O—(CₚHₚ₂—O)ₓ-L; —(CₚH₂ₚ—O)ₓ-L; wherein p represents a natural number from 1 up to 4 and x represents an integer from 1 up to 10, and
wherein Y and Z, independently from each other, have the meanings described for L, and wherein terminal amino groups are optionally present in the form of their hydrohalides, acetamides, mono-, di- or trihaloacetamides, wherein “halo-”, and resp., “halide” means fluorine and/or chlorine and/or bromine and/or iodine,
and/or wherein R1 and R2 represent, independently from each other, a fatty acid residue
—CH₂—(CᵣH₂ᵣ)—COOH, —CH₂—(CᵣH₂ᵣ₋₂)—COOH, —CH₂—(CᵣH₂ᵣ₋₄)—COOH, —CH₂—(CᵣH₂ᵣ₋₆)—COOH, —CH₂—(CᵣH₂ᵣ₋₈)—COOH or an adamantane-1-yl-ester of one of these fatty acid residues and
r represents an integer from 10 up to 18,
and/or wherein R1 and R2, independently from each other, represent the residue R6 of an amino acid, 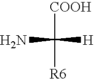
wherein R6 preferentially represents benzyl-, 4-hydroxy-benzyl-, -(1 H-indolyl)-methyl-, (1 H-imidazolyl-methyl-, 4-amino-butyl-, (3-guanidyl)-propyl, (2-methylthio)-ethyl, hydroxymethyl-, (R)-(1-hydroxy)-ethyl, (S)-(1-hydroxy)-ethyl, (2-carboxy)-ethyl-,(R)-(2-carbamoyl-1-methyl)-ethyl, (S)-(2-carbamoyl-1-methyl)-ethyl-, carboxymethyl-, thiomethyl-, (2-carbamoyl)-ethyl-, (carbamoyl)-methyl-, selenomethyl-, (3-amino)-propyl-, 2-aminophenyl-2-oxo-ethyl-
R3=—H or L, wherein L has the meanings listed under R1, R2 and wherein L optionally carries one up to three substituents selected from the group comprising: —F; —Cl; —Br; —I; —OH; —O—(C₁-C₁₀-alkyl); —SH; —S—(C₁-C₁₀-alkyl); —SO₃H; —CN; —COOH; —COO—(C₁-C₁₀-alkyl); —O—(C═O)—(C₁-C₁₀-alkyl); —(C₁-C₁₀-alkyl); —CONH₂; —CONH(C₁-C₁₀-alkyl); —CON(C₁-C₁₀-alkyl)₂, wherein the two alkyl groups are identical or different; —NH₂, —NH(C₁-C₁₀-alkyl); —N(C₁-C₁₀-alkyl)₂, wherein the two alkyl groups are identical or different, and wherein the alkyl groups are linear or branched,
A=being a single bond or
—(C═O)-(AA)ₘ-, wherein every unit of M represents a natural or non-natural α-, β-, γ- or δ-amino acid in the D or L configuration,
m represents an integer from 0 up to 10,
the amino acids within the peptide chain m are identical or different, and
wherein the N terminus of (AA)ₘ is bound to the C═O-group listed in the definition of A and the C terminus of (AA)ₘ is bound to R4,
or A represents a 2- up to 20-membered depsipeptide, wherein every amino acid within the depsipeptide represents a natural or non-natural α-, β-, γ- or δ-amino acid in the D or L configuration and wherein every carboxylic acid within the depsipeptide represents an aliphatic, aromatic, or araliphatic carboxylic acid with 1 up to 10 carbon atoms, wherein aliphatic groups are linear, cyclic, or branched, and wherein every carboxylic acid optionally carries one up to three substituents selected from the group comprising: —OH, —(C═O)—, —COOH, —F, —Cl, —Br, —I, —NH₂, —SH, —S—S—,
R4=—H or L, wherein L has the meanings listed under R1, R2; and wherein L optionally carries one up to three substituents selected from the group comprising: —F; —Cl; —Br; —I; —OH; —O—(C₁-C₁₀-alkyl); —SH; —S—(C₁-C₁₀-alkyl); —SO₃H; —CN; —COOH; —COO—(C₁-C₁₀-alkyl); —O—(C═O)—(C₁-C₁₀-alkyl); —(C₁-C₁₀-alkyl); —CONH₂—CONH(C₁-C₁₀-alkyl); —CON(C₁-C₁₀-alkyl)₂, wherein the two alkyl groups are identical or different, —NH₂, —NH(C₁-C₁₀-alkyl); —N(C₁-C₁₀-alkyl)₂, wherein the two alkyl groups are identical or different; and wherein the alkyl groups are linear or branched; 1-adamantyle containing optionally one up to three substituents selected from the group comprising: —F; —Cl; —Br; —I; —OH; —O—(C₁-C₁₀-alkyl); —SH; —S—(C₁-C₁₀-alkyl); —SO₃H; —CN; —COOH; —COO—(C₁-C₁₀-alkyl); —O—(C═O)—(C₁-C₁₀-alkyl); —(C₁-C₁₀)-alkyl); —CONH₂; —CONH(C₁-C₁₀-alkyl); —CON(C₁-C₁₀-alkyl)₂, wherein the two alkyl groups are identical or different; —NH₂, —NH(C₁-C₁₀-alkyl); —N(C₁-C₁₀-alkyl)₂, wherein the two alkyl groups are identical or different, and the alkyl groups are linear or branched, adamantly comprising optionally one up to three substituents selected from the group comprising: —F; —Cl; —Br; —I; —OH; —O—(C₁-C₁₀-alkyl); —SH; —S—(C₁-C₁₀-alkyl); —SO₃H; —CN; —COOH; —COO—(C₁-C₁₀-alkyl); —CONH₂; —CONH(C₁-C₁₀-alkyl); —CON(C₁-C₁₀-alkyl)₂, both alkyl groups being identical or different; —NH₂, —NH(C₁-C₁₀-alkyl); —N(C₁-C₁₀-alkyl)₂, the two alkyl groups being identical or different and said alkyl groups being linear or branched and have the meanings listed under R1 and R2, if A=—(C=O)-(AA)ₘ-
or
R4 has the meanings listed under R1 and R2 if A=single bond,
B=single bond
or (AA)_{q}, wherein every amino acid unit represents a natural or non-natural α-, β-, γ- or δ-amino acid in the D or L configuration,
q represents an integer from 0 up to 10,
the amino acids within the peptide chain q are identical or different, and wherein the C terminus of (AA)_{q} is bound to the nitrogen atom linked to the adamantyl residue and the N terminus of (AA)_{q} being bound to R5,
or B represents a 2- up to 10-membered depsipeptide, wherein every amino acid within the depsipeptide represents a natural or non-natural α-, β-, γ- or δ-amino acid in the D or L configuration and wherein every carboxylic acid within the depsipeptide represents an aliphatic, -aromatic or araliphatic carboxylic acid with 1 up to 10 carbon atoms, wherein aliphatic groups are linear, cyclic or branched, and wherein every carboxylic acid optionally carries one up to three substituents selected from the group comprising: —OH, —(C=O)—, —COOH, —F, —Cl, —Br, —I, —NH₂, —SH, —S—S—,
R5=—H or L, wherein L has the meanings listed under R1, R2; and wherein L optionally carries one up to three substituents selected from the group comprising: —F; —Cl; —Br; —I; —OH; —O—(C₁C₁₀-alkyl); —SH; —S—(C₁-C₁₀-alkyl); —SO₃H; —CN; —COOH; —COO—(C₁-C₁₀-alkyl); —O—(C═O)—(C₁-C₁₀-alkyl); —(C₁-C₁₀-alkyl); —CONH₂; —CONH(C₁-C₁₀-alkyl); —CON(C₁-C₁₀-alkyl)₂, wherein the two alkyl groups are identical or different; —NH₂, —NH(C₁-C₁₀-alkyl); —N(C₁-C₁₀-alkyl)₂, wherein the two alkyl groups are identical or different, and wherein the alkyl groups are linear or branched;
or
—SO₂L, —(C═O)-L; —COOL; —(CₚH₂ₚ)ₓ—O-L; —(CₚH₂ₚ—O)ₓ-L; wherein p, x and L have the meanings listed under R1 and R2, or
1-adamantyl comprising optionally one up to three substituents selected from the group comprising: —F; —Cl; —Br; —I; —OH; —O—(C₁-C₁₀-alkyl); —SH; —S—(C₁-C₁₀-alkyl); —SO₃H; —CN; —COOH; —COO—(C₁-C₁₀-alkyl); —CONH₂; —CONH(C₁-C₁₀-alkyl); —CON(C₁-C₁₀-alkyl)₂, wherein the two alkyl groups are identical or different; —NH₂, —NH(C₁-C₁₀-alkyl); —N(C₁-C₁₀-alkyl)₂, wherein the two alkyl groups are identical or different, and the alkyl groups have the meaning listed under R1 and R2,
and
n=1-40 and an integer in case of an open-chained compound, wherein R1, R2, R3, A and B are identical or different in every unit n and at least one of the groups R1, R2 and R4 is different from hydrogen,
or
R4, R5=single bonds and n =1-25 in the case of a cyclic oligopeptide, wherein R1, R2, R3, A and B are identical or different in every unit n, wherein B is a sequence (AA)_{z} of natural or non-natural α-, β-, γ- or δ-amino acids AA in the D or L configuration or a 1-25-membered depsipeptide, wherein every amino acid within the depsipeptide is a natural or non-natural α-, β-, γ- or δ-amino acid in the D or L configuration and wherein every carboxylic acid within the depsipeptide is an aliphatic, aromatic or araliphatic carboxylic acid with 1 up to 10 carbon atoms, wherein aliphatic groups are linear, cyclic, or branched, and wherein every carboxylic acid optionally carries one up to three substituents selected from the group comprising: —OH, —(C═O)—, —COOH, —F, —Cl, —Br, —I, —NH₂, —SH, —S—S—, and z, if n=1, is an integer from 3 up to 25, or
z, if n=2, is an integer from 2 up to 25, or
B, if n=1, is a 3-aminoadamantane-carboxylic acid of the present invention,
and with the proviso that n=1 and both A and B are single bonds, such compounds are excluded wherein the following combinations of residues of R1, R2, R3, R4 and R5 are represented:
compounds wherein R1, R2 and R3 are hydrogen, R4 is a carboxyl group and R5 is hydrogen or an acetyl group,
a compound in which R1, R2 and R3 are hydrogen, R4 is a carboxymethyl group and R5 is an acetyl group,
a compound in which R1 and R2 are methyl groups and R3, R4 and R5 are hydrogen,
as well as compounds in which R1 and R2 have been selected, independently from each other, from —H, —F, —Cl, —Br, —I, —CN, —COOH, —CH₃, —CH₂CH₃, —OCH₃, —OCH₂CH₃, —CH₂CH₂CH₃, —CH(CH₃)₂,
—CH₃—O—(C═O)—, CH₃—O—(C═O)—and CH₃CH₂—O—(C═O)—; R3 is hydrogen and R4 is a carboxyl group and R5 was selected from the group comprising: 3-formylamino-2-hydroxy-benzoyl-, 3-acetylamino-2-hydroxy-benzoyl-, 2-hydroxy-3-propionylamino-benzoyl-, 2-butyrylamino-2-hydroxy-benzoyl-, 2-hydroxy-3-isobutyrylamino-, 2-hydroxy-3-pentanoylamino-benzoyl-, 2-hydroxy-3-(3-methyl-butyrylamino-) benzoyl-, 2-hydroxy-3-(2-methyl-butyrylamino)-benzoyl-, 3-(2 ,2-dimethyl-propionylamino)-2-hydroxy-benzoyl-, 2-hydroxy-3-methoxycarbonylamino-benzoyl-, 2-hydroxy-3-ethoxycarbonylamino-benzoyl-, 2-hydroxy-3-propoxycarbonylamino-benzoyl-, 2-hydroxy-3-isopropoxycarbonylamino-benzoyl-,
and furthermore, such compounds are excluded wherein A and B are single bonds,
n=1, R3 and R5 have been selected, independently from each other, from H, aryl or heteroaryl with up to 7 ring elements, C₁—C₁₀-alkyl, C₁-C₁₀-alkenyl and C₁-C₁₀-alkinyl, wherein the alkyl-, alkenyl- and alkinyl residues are branched, linear, or cyclic and may have been substituted with halogen, aryl or heteroaryl comprising up to 7 ring elements,
and R1, R2 and R4 have been selected, independently from each other, from —NR19R20, H, aryl or heteroaryl with up to 7 ring elements, C₁-C₁₀-alkyl, C₁-C₁₀-alkenyl and C₁-C₁₀-alkinyl, wherein the alkyl-, alkenyl- and alkinyl residues are branched, linear or cyclic and being optionally substituted with halogen, aryl or heteroaryl with up to 7 ring elements, and R19 and R20 being selected, independently from each other, from H, aryl or heteroaryl with up to 7 ring elements, C₁-C₁₀-alkyl, C₁-C₁₀-alkenyl and C₁-C₁₀-alkinyl, wherein the alkyl-, alkenyl- and alkinyl residues are branched, linear or cyclic and are optionally substituted with halogen, aryl or heteroaryl with up to 7 ring elements, or R1 and R20, together with the nitrogen atom, form a heterocyclic group with up to 7 ring elements,
and furthermore, such compounds are excluded wherein A and B are single bonds,
n=1, residues R1 and R2 represent lower straight-chained or branched alkyl residues, R3 represents hydrogen, a lower straight-chained or branched alkyl residue and residue R5 represents a hydrogen or methyl group.

2. A compound according to claim 1 in which A represents a single bond, R4=—COOH, —COO—, or —COOL,
L=-alkyl, -alkenyl, -alkinyl, -cycloalkyl, -cycloalkenyl, -heterocycloalkyl, -heterocycloalkenyl, -aryl, -heteroaryl, -alkylaryl, -alkylheteroaryl, -alkylcycloalkyl, -alkylheterocycloalkyl, -alkenylcycloalkyl, -alkenylheterocycloalkyl,
wherein -alkyl represents a group containing 1 up to 10 carbon atoms, preferentially methyl, ethyl, propyl, isopropyl, 1-butyl, 2-butyl, (2-methyl-)propyl, tert.-butyl and
-alkenyl and -alkinyl represent a monounsaturated or polyunsaturated group with 2 up to 10 carbon atoms which, in the case of the alkenyl, contains at least one —C═C-bond and, in the case of the alkinyl, contains at least one —C≡C-bond;
-alkyl, -alkenyl, resp., -alkinyl are linear or branched, -cycloalkyl and -cycloalkenyl represent a group with 3 up to 20 carbon atoms; the heterocyclic groups represent a residue with 1 up to 20 carbon atoms, wherein up to 5 carbon atoms have been substituted by heteroatoms selected from the following: nitrogen, oxygen, sulphur, phosphorus; -aryl represents an aromatic residue with 5 up to 20 carbon atoms and heteroaryl represents a corresponding aromatic residue wherein up to 5 carbon atoms have been substituted by heteroatoms selected from the following: nitrogen, oxygen, sulphur, phosphorus,
wherein L optionally carries 1 up to 3 substituents selected from the following: F; Cl; Br; I; —OH; —O—(C₁-C₁₀-alkyl); —SH; —S—(C₁-C₁₀-alkyl); —SO₃H; —CN; —COOH; —COO—(C₁-C₁₀-alkyl); —O—(C═O)——(C₁-C₁₀-alkyl); —CONH₂; —CONH(C₁-C₁₀-alkyl); —CON(C₁-C₁₀-alkyl)₂, wherein the two alkyl groups are identical or different; —NH₂, —NH(C₁-C₁₀-alkyl); —N(C₁-C₁₀-alkyl)₂, wherein the two alkyl groups are identical or different, and wherein alkyl groups are linear or branched,
and wherein n=1.

3. A compound in accordance with claim 2 wherein R4 represents a COOH group.

4. A compound in accordance with claim 1 wherein A=single bond, R4=H, F, Cl, Br, I; or L, wherein L represents -alkyl, -alkenyl, -alkinyl, -cycloalkyl, -cycloalkenyl, -heterocycloalkyl, -heterocycloalkenyl, -aryl, -heteroaryl, -alkylaryl, -alkylheteroaryl, -alkylcycloalkyl, -alkylheterocycloalkyl, -alkenylcycloalkyl, or -alkenylheterocycloalkyl, and wherein -alkyl represents a group with 1 up to 10 carbon atoms, preferentially methyl, ethyl, propyl, isopropyl, 1-butyl, 2-butyl, (2-methyl-)propyl, tert.-butyl, and wherein -alkenyl and -alkinyl represent a monounsaturated or polyunsaturated group with 2 up to 10 carbon atoms which contains, in the case of alkenyl, at least one —C═C-bond and, in the case of alkinyl, at least one —C≡C-bond;
-alkyl, -alkenyl, and resp., -alkinyl are linear or branched, -cycloalkyl and -cycloalkenyl represent a group with 3 up to 20 carbon atoms; the heterocyclic groups represent a residue with 1 up to 20 carbon atoms, wherein up to 5 carbon atoms have been substituted by heteroatoms selected from the following: nitrogen, oxygen, sulphur, phosphorus,
-aryl represents an aromatic residue with 5 up to 20 carbon atoms and heteroaryl represents a corresponding aromatic residue wherein up to 5 carbon atoms have been substituted by heteroatoms selected from the following: nitrogen, oxygen, sulphur, phosphorus, wherein L optionally carries 1 up to 3 substituents selected from the following: F; Cl; Br; I; —OH; —O—(C₁-C₁₀-alkyl); —SH; —S—(C₁-C₁₀-alkyl); —SO₃H; —CN; —COOH; —COO—(C₁-C₁₀-alkyl); —O—(C═O)——(C₁-C₁₀-alkyl); —CONH₂; —CONH(C₁-C₁₀-alkyl); —CON(C₁-C₁₀-alkyl)₂, wherein the two alkyl groups are identical or different; —NH₂, —NH(C₁-C₁₀-alkyl); —N(C₁-C₁₀-alkyl)₂, wherein the two alkyl groups are identical or different; and wherein alkyl groups are linear or branched,
or R4 represents —OH, —O—, —OL, —SH, —S—, —SL, —SOH, —SO₂H, —SO₃H, —(S═O)-L, —SO₂L, —NO, —NO₂, —C=N, —C≡N-L, —N—C, —N═C-L, —NH₂, —NHL, —NH₂L⁺, —NYZ, —NHYZ⁺, —CHO, —O(CHO), —O(C═O)L, —CONH₂, —CONHL, —CONYZ, —NHCOOH, —NLCOOH, —NLCOOL, —NHCOOL, —NH—(C═O)L, —NH—(C═N—H)—NH₂, —NH—(C═N—H)—NYZ, —NY—(C═N—Z)—NHL, —NH—(C═N—H)—NHL, —SO₂—NH-L, —SO₂—NH₂, —SO₂—NYZ, —NY—SO₂Z, —O—(CₚH₂ₚ)ₓ—O-L; —(CₚH₂ₚ)ₓ—O-L; —O—(CₚH₂ₚ—O)ₓ-L; or —(CₚH₂ₚ—O)ₓ-L; wherein p represents a natural number from 1 up to 4 and x represents a natural number from 1 up to 10, and wherein Y and Z, independently from each other, have the meanings described for L, and wherein terminal amino groups are optionally present in the form of their hydrohalides, acetamides, mono-, di- or trihaloacetamides, wherein “halo-” resp. “halide” means fluorine and/or chlorine and/or bromine and/or iodine.

5. A compound according to claim 1 and being a linear oligopeptide, wherein n represents a natural number from 1 up to 40, preferentially 16, 17, or 18.

6. A compound according to claim 1 and being a cyclic oligopeptide, wherein R4 and R5 represent single bonds, n represents a natural number from 1 up to 25, A and B are identical or different in every unit, and wherein B represents a sequence (AA)_{z} of natural or non-natural α-, β-, γ- or δ-amino acids AA in the D or L configuration or a 1-25-membered depsipeptide, wherein every amino acid within the depsipeptide represents a natural or non-natural α-, β-, γ- or δ-amino acid in the D or L configuration and wherein every carboxylic acid within the depsipeptide represents an aliphatic, aromatic or araliphatic carboxylic acid with 1 up to 10 carbon atoms, wherein aliphatic groups are linear, cyclic or branched and wherein every carboxylic acid optionally carries one up to three substituents selected from the following: —OH, —(C═O)—, —COOH, —F, —Cl, —Br, —I, —NH₂, —SH, —S—S—, and wherein z represents a natural number from 3 up to 25.

7. A compound according to claim 5, wherein n=1 and z represents a natural number from 3 up to 25.

8. A compound according to claim 5, wherein n=2 and z represents a natural number from 2 up to 25.

9. A compound according to claim 5, wherein n=1 and B is a 3-aminoadamantane carboxylic acid.

10. A process for the production of compounds according to claim 3, characterized by the steps:
a) reacting of a 1,3-disubstituted adamantane derivative with a first halogen source under phase transfer catalytic conditions;
b) reacting of a 1,3-disubstituted adamantane derivative with a second halogen source under phase transfer catalytic conditions, wherein the halogens of the first and second halogen source are different;
c) reacting of the 1,3-dihalogenoadamantane derivative with a one-electron oxidant and a nitrile;
d) reacting with water;
e) reacting of the 1-halogeno-3-aminoadamantane-derivative with carbon monoxide;
f) purification of the final product.

11. A method for the production of compounds according to claim 10, wherein under phase transfer catalytic conditions is understood the dissolution of the educts in an apolar aprotic solvent and the subsequent adding of inorganic base and a halogen source.

12. A method for the production of compounds according to claim 11, wherein the apolar, aprotic solvent has been selected from one of the following: fluorobenzene, methylene chloride, trifluoromethylbenzene, benzene, 1, 3-di-(trifluoromethyl)-benzene, chloro benzene, bromo benzene, tert.-butylmethylether, cycloalkanes with 5 to 8 C-atoms.

13. A method for the production of compounds according to claim 11, wherein the inorganic base has been selected from one of the following: NaOH, KOH, CsOH, Ba(OH)₂, NaClO₃, wherein the base is added in a solid form or in the form of an aqueous solution.

14. A method for the production of compounds according to claim 10, wherein the one-electron oxidant has been selected from one of the following: NOBF₄ and NOSF₆.

15. A method for the production of compounds according to claim 10, wherein the reaction according to step d) is carried out with an alcohol, rather than water.

16. A method for the production of compounds according to claim 10, wherein after completion of step e) the following steps are carried out:
a) masking of amino- and/or carboxyl groups by suitable protective groups
b) reaction with a halogen source under phase transfer catalytic conditions
c) reaction with a nucleophile selected from one of the following: Grignard compounds, lithium-organic compounds, alkali sulphites, saline cyanides,
d) further reaction into residues R1 or R2, wherein R1 and R2 are defined as in claim 3,
e) purification of the final product

17. A method for the production of compounds according to claim 16, wherein the nucleophile according to step c) is
a nitrile which reacts, according to claim 16, step d), into a residue R1 or R2 selected from one of the following: carbamoyls, carboxyl derivatives, aminomethylderivatives, ketone derivatives, esters or amides, or
a Grignard or lithium-organic compound which reacts, according to claim 16, step d), into a residue R1 or R2 selected from one of the following: carboxylic acids, ketones, esters, primary alcohols, secondary alcohols, tertiary alcohols, alkyl compounds, or
an alkali sulphite which reacts, according to claim 16, step d), into a residue R1 or R2 selected from one of the following: sulphonic acids and sulphonic acid derivatives, or
a trimethylsilyl compound which reacts, according to claim 16, step d), into a residue R1 or R2 selected from one of the following: aliphatic, aromatic or araliphatic,
wherein R1 and R2 are defined as in claim 3.

18. A method for the production of compounds according to claim 16, wherein after completion of step b) wherein a 1,3- disubstitued adamantine is reacted with a second halogen source, a second reaction with a halogen source and a phase transfer catalyst is carried out, wherein the halogens of the first and second halogen source are different and steps c) of the reaction with a halogen source and d) of the reaction with a nucleophile are repeated once.

19. A method for the production of 3,5-substituted 1-aminoadamantane derivatives, characterized by the steps:
a) suspending of a 1,3-disubstituted adamantane derivative in HNO₃ and H₂SO₄;
b) adding of oleum;
c) reacting with a nitrile;
d) reacting with an alcohol or water;
e) purification of the final product

20. A method for the production of 3,5-substituted 1-aminoadamantane derivatives according to claim 19, wherein the 1,3-disubstituted adamantane derivative is suspended or dissolved in 100% HNO₃; preferentially, a solution of the 1,3-disubstituted adamantane derivative is produced in HNO₃ and the adding of H₂SO₄ and oleum is foregone.

21. A method in accordance with claim 10, wherein the purification of the final product is carried out via recrystallisation or column chromatography.

22. A method in accordance with claim 10, wherein the purified final product is subjected to an enantiomer separation, preferentially an enantiomer separation via chiral HPLC or fractionated cocrystallisation with quinine.

23. A method for the producing compounds as defined in claim 5, characterized by the following steps:
a) masking of all non-C-terminal carboxyl groups and non-N-terminal amino groups with protective groups;
b) activating the C-terminal carboxyl group by an activation reagent selected from the group comprising: DIC, DCC, EDC, FmocOPfp, PyClop, HBTU, HATU, HOSu, TBTU, T3P, BopCl, 3-Cl-1-pyridiniumiodide;
c) coupling of C-terminal activated and N-terminal unprotected amino acid;
d) removing of the protective groups;
e) repeating of steps a) to d) until the desired chain length is achieved;
f) purification of the final product.

24. A method according to claim 23, wherein the purification of the final products is carried out via recrystallisation or column chromatography.

25. Utilisation of compounds in accordance with claim 1 and utilisation of their pharmaceutically acceptable salts, esters and/or amides for the production of a drug.

26. Utilisation of compounds according to claim 25 for the production of a drug for the therapy, diagnostics and prophylaxis of viral diseases.

27. The utilisation of compounds according to claim 25 for the production of a drug for the therapy, diagnostics and prophylaxis of diseases linked with a dysfunction of the GABA system.

28. Utilisation of compounds according to claim 25 and utilisation of their pharmaceutically acceptable salts, esters and/or amides for the production of a drug for the treatment of Morbus Parkinson, Chorea Huntington, Morbus Alzheimer, autism, tinnitus, Tourette Syndrome, hypertension, sleep disorders, ADHD, psychoses, panic and anxiety disorders, posttraumatic stress syndrome, manic depressive disorders and schizophrenia.

29. Utilisation of compounds according to claim 6 and utilisation of their pharmaceutically acceptable salts, esters and/or amides for the production of a drug for the therapy, diagnostics and prophylaxis of diseases caused by a dysfunction of the natural ion channels or in which there is a deficit of natural ion channels.

30. Utilisation of compounds according to claim 5 and utilisation of their pharmaceutically acceptable salts, esters and/or amides for the production of a drug for the treatment of cystic fibrosis and mucoviscidosis.

31. Utilisation of compounds according to claim 1 and utilisation of their pharmaceutically acceptable salts, esters and/or amides for the production of a drug for the therapy, diagnosis and prophylaxis of infections caused by plasmodia.

32. Utilisation of compounds according to claim 1 and utilisation of their pharmaceutically acceptable salts, esters and/or amides for the production of a drug for the therapy, diagnosis and prophylaxis of malaria diseases caused by plasmodia selected from the group comprised of Plasmodium falciparum, Plasmodium ovale, Plasmodium vivax and Plasmodium malariae.

33. Pharmaceutically acceptable composition containing compounds according to claim 1 as active components, and a physiologically acceptable carrier.

34. Pharmaceutically acceptable composition according to claim 31, comprising its application in oral, rectal, parenteral, intravenous, intramuscular, subcutaneous, intracisternal, intravaginal, intraperitoneal, intravascular, intrathecal, intravesical, topic, local or spray form (aerosol).

35. Utilisation of compositions according to claim 1 as a component of a peptidic catalyst.
